SEQUENCE LISTING (20 sequences)

SEQ ID NO: 1 - DNA, 1070 bp, synthetic construct
  misc_feature 1..1070: note = Expressionskonstrukt
  source 1..1070: mol_type = other DNA
gccctgcaggtaaaaaaaggattgtgacttctccagcaacactgaaagttctcaacgcctacttggataaccccactccaaccctggaggaggcaattgaggtgttcaccccgctgaccgtgggtgaatacgatgacgtgcacatcgcagcgctgcttgccaccatccgtactcgcggtgagcagttcgctgatattgccggcgctgccaaggcgttcctcgcggcggctcgtccgttcccgattactggcgcaggtttgctagattccgctggtactggtggcgacggtgccaacaccatcaacatcaccaccggcgcatccctgatcgcagcatccggtggagtgaagctggttaagcacggcaaccgttcggtgagctccaagtccggctccgccgatgtgctggaagcgctgaatattcctttgggccttgatgtggatcgtgctgtgaagtggttcgaagcgtccaacttcaccttcctgttcgcacctgcgtacaaccctgcgattgcgcatgtgcagccggttcgccaggcgctgaaattccccaccatcttcaacacgcttggaccattgctgtccccggcgcgcccggagcgtcagatcatgggcgtggccaatgccaatcatggacagctcatcgccgaggtcttccgcgagttgggccgtacacgcgcgcttgttgtgcatggcgcaggcaccgatgagatcgcagtccacggcaccaccttggtgtgggagcttaaagaagacggcaccatcgagcattacaccatcgagcctgaggaccttggccttggccgctacacccttgaggatctcgtaggtggcctcggcactgagaacgccgaagctatgcgcgctactttcgcgggcaccggccctgatgcacaccgtgatgcgttggctgcgtccgcaggtgcgatgttctacctcaacggcgatgtcgactccttgaaagatggtgcacaaaaggcgctttccttgcttgccgacggcaccacccaggcatggttggccaagcacgaagagatcgattactcagaaaaggagtcttccaatgactag

SEQ ID NO: 2 - DNA, 1070 bp, synthetic construct
  misc_feature 1..1070: note = Expressionskonstrukt
  source 1..1070: mol_type = other DNA
gccctgcaggtaaaaaaaggattttgacttctccagcaacactgaaagttctcaacgcctacttggataaccccactccaaccctggaggaggcaattgaggtgttcaccccgctgaccgtgggtgaatacgatgacgtgcacatcgcagcgctgcttgccaccatccgtactcgcggtgagcagttcgctgatattgccggcgctgccaaggcgttcctcgcggcggctcgtccgttcccgattactggcgcaggtttgctagattccgctggtactggtggcgacggtgccaacaccatcaacatcaccaccggcgcatccctgatcgcagcatccggtggagtgaagctggttaagcacggcaaccgttcggtgagctccaagtccggctccgccgatgtgctggaagcgctgaatattcctttgggccttgatgtggatcgtgctgtgaagtggttcgaagcgtccaacttcaccttcctgttcgcacctgcgtacaaccctgcgattgcgcatgtgcagccggttcgccaggcgctgaaattccccaccatcttcaacacgcttggaccattgctgtccccggcgcgcccggagcgtcagatcatgggcgtggccaatgccaatcatggacagctcatcgccgaggtcttccgcgagttgggccgtacacgcgcgcttgttgtgcatggcgcaggcaccgatgagatcgcagtccacggcaccaccttggtgtgggagcttaaagaagacggcaccatcgagcattacaccatcgagcctgaggaccttggccttggccgctacacccttgaggatctcgtaggtggcctcggcactgagaacgccgaagctatgcgcgctactttcgcgggcaccggccctgatgcacaccgtgatgcgttggctgcgtccgcaggtgcgatgttctacctcaacggcgatgtcgactccttgaaagatggtgcacaaaaggcgctttccttgcttgccgacggcaccacccaggcatggttggccaagcacgaagagatcgattactcagaaaaggagtcttccaatgactag

SEQ ID NO: 3 - protein, 348 aa, Corynebacterium glutamicum
MTSPATLKVLNAYLDNPTPTLEEAIEVFTPLTVGEYDDVHIAALLATIRTRGEQFADIAGAAKAFLAAARPFPITGAGLLDSAGTGGDGANTINITTGASLIAASGGVKLVKHGNRSVSSKSGSADVLEALNIPLGLDVDRAVKWFEASNFTFLFAPAYNPAIAHVQPVRQALKFPTIFNTLGPLLSPARPERQIMGVANANHGQLIAEVFRELGRTRALVVHGAGTDEIAVHGTTLVWELKEDGTIEHYTIEPEDLGLGRYTLEDLVGGLGTENAEAMRATFAGTGPDAHRDALAASAGAMFYLNGDVDSLKDGAQKALSLLADGTTQAWLAKHEEIDYSEKESSND

SEQ ID NO: 4 - protein, 919 aa, Corynebacterium glutamicum
MTDFLRDDIRFLGQILGEVIAEQEGQEVYELVEQARLTSFDIAKGNAEMDSLVQVFDGITPAKATPIARAFSHFALLANLAEDLYDEELREQALDAGDTPPDSTLDATWLKLNEGNVGAEAVADVLRNAEVAPVLTAHPTETRRRTVFDAQKWITTHMRERHALQSAEPTARTQSKLDEIEKNIRRRITILWQTALIRVARPRIEDEIEVGLRYYKLSLLEEIPRINRDVAVELRERFGEGVPLKPVVKPGSWIGGDHDGNPYVTAETVEYSTHRAAETVLKYYARQLHSLEHELSLSDRMNKVTPQLLALADAGHNDVPSRVDEPYRRAVHGVRGRILATTAELIGEDAVEGVWFKVFTPYASPEEFLNDALTIDHSLRESKDVLIADDRLSVLISAIESFGFNLYALDLRQNSESYEDVLTELFERAQVTANYRELSEAEKLEVLLKELRSPRPLIPHGSDEYSEVTDRELGIFRTASEAVKKFGPRMVPHCIISMASSVTDVLEPMVLLKEFGLIAANGDNPRGTVDVIPLFETIEDLQAGAGILDELWKIDLYRNYLLQRDNVQEVMLGYSDSNKDGGYFSANWALYDAELQLVELCRSAGVKLRLFHGRGGTVGRGGGPSYDAILAQPRGAVQGSVRITEQGEIISAKYGNPETARRNLEALVSATLEASLLDVSELTDHQRAYDIMSEISELSLKKYASLVHEDQGFIDYFTQSTPLQEIGSLNIGSRPSSRKQTSSVEDLRAIPWVLSWSQSRVMLPGWFGVGTALEQWIGEGEQATQRIAELQTLNESWPFFTSVLDNMAQVMSKAELRLAKLYADLIPDTEVAERVYSVIREEYFLTKKMFCVITGSDDLLDDNPLLARSVQRRYPYLLPLNVIQVEMMRRYRKGDQSEQVSRNIQLTMNGLSTALRNSG

SEQ ID NO: 5 - DNA, 2760 bp, Corynebacterium glutamicum
  source 1..2760: mol_type = genomic DNA
atgactgattttttacgcgatgacatcaggttcctcggtcaaatcctcggtgaggtaattgcggaacaagaaggccaggaggtttatgaactggtcgaacaagcgcgcctgacttcttttgatatcgccaagggcaacgccgaaatggatagcctggttcaggttttcgacggcattactccagccaaggcaacaccgattgctcgcgcattttcccacttcgctctgctggctaacctggcggaagacctctacgatgaagagcttcgtgaacaggctctcgatgcaggcgacacccctccggacagcactcttgatgccacctggctgaaactcaatgagggcaatgttggcgcagaagctgtggccgatgtgctgcgcaatgctgaggtggcgccggttctgactgcgcacccaactgagactcgccgccgcactgtttttgatgcgcaaaagtggatcaccacccacatgcgtgaacgccacgctttgcagtctgcggagcctaccgctcgtacgcaaagcaagttggatgagatcgagaagaacatccgccgtcgcatcaccattttgtggcagaccgcgttgattcgtgtggcccgcccacgtatcgaggacgagatcgaagtagggctgcgctactacaagctgagccttttggaagagattccacgtatcaaccgtgatgtggctgttgagcttcgtgagcgtttcggcgagggtgttcctttgaagcccgtggtcaagccaggttcctggattggtggagaccacgacggtaacccttatgtcaccgcggaaacagttgagtattccactcaccgcgctgcggaaaccgtgctcaagtactatgcacgccagctgcattccctcgagcatgagctcagcctgtcggaccgcatgaataaggtcaccccgcagctgcttgcgctggcagatgcagggcacaacgacgtgccaagccgcgtggatgagccttatcgacgcgccgtccatggcgttcgcggacgtatcctcgcgacgacggccgagctgatcggcgaggacgccgttgagggcgtgtggttcaaggtctttactccatacgcatctccggaagaattcttaaacgatgcgttgaccattgatcattctctgcgtgaatccaaggacgttctcattgccgatgatcgtttgtctgtgctgatttctgccatcgagagctttggattcaacctttacgcactggatctgcgccaaaactccgaaagctacgaggacgtcctcaccgagcttttcgaacgcgcccaagtcaccgcaaactaccgcgagctgtctgaagcagagaagcttgaggtgctgctgaaggaactgcgcagccctcgtccgctgatcccgcacggttcagatgaatacagcgaggtcaccgaccgcgagctcggcatcttccgcaccgcgtcggaggctgttaagaaattcgggccacggatggtgcctcactgcatcatctccatggcatcatcggtcaccgatgtgctcgagccgatggtgttgctcaaggaattcggactcatcgcagccaacggcgacaacccacgcggcaccgtcgatgtcatcccactgttcgaaaccatcgaagatctccaggccggcgccggaatcctcgacgaactgtggaaaattgatctctaccgcaactacctcctgcagcgcgacaacgtccaggaagtcatgctcggttactccgattccaacaaggatggcggatatttctccgcaaactgggcgctttacgacgcggaactgcagctcgtcgaactatgccgatcagccggggtcaagcttcgcctgttccacggccgtggtggcaccgtcggccgcggtggcggaccttcctacgacgcgattcttgcccagcccaggggggctgtccaaggttccgtgcgcatcaccgagcagggcgagatcatctccgctaagtacggcaaccccgaaaccgcgcgccgaaacctcgaagccctggtctcagccacgcttgaggcatcgcttctcgacgtctccgaactcaccgatcaccaacgcgcgtacgacatcatgagtgagatctctgagctcagcttgaagaagtacgcctccttggtgcacgaggatcaaggcttcatcgattacttcacccagtccacgccgctgcaggagattggatccctcaacatcggatccaggccttcctcacgcaagcagacctcctcggtggaagatttgcgagccatcccatgggtgctcagctggtcacagtctcgtgtcatgctgccaggctggtttggtgtcggaaccgcattagagcagtggattggcgaaggggagcaggccacccaacgcattgccgagctgcaaacactcaatgagtcctggccatttttcacctcagtgttggataacatggctcaggtgatgtccaaggcagagctgcgtttggcaaagctctacgcagacctgatcccagatacggaagtagccgagcgagtctattccgtcatccgcgaggagtacttcctgaccaagaagatgttctgcgtaatcaccggctctgatgatctgcttgatgacaacccacttctcgcacgctctgtccagcgccgatacccctacctgcttccactcaacgtgatccaggtagagatgatgcgacgctaccgaaaaggcgaccaaagcgagcaagtgtcccgcaacattcagctgaccatgaacggtctttccactgcgctgcgcaactccggctag

SEQ ID NO: 6 - protein, 185 aa, Corynebacterium glutamicum
MNDQIHLDHQSDDTSECSCPIVVLVGLPGAGKSTIGRRLARALNTELVDSDELIERATGKACGAVFSELGEPAFRELEAIHVAEALKSSGVVSLGGGSVLTESTRELLKGQDVVWIDVPVEEGIRRTANERSRPVLQAADPAEHYRNLVKVRTPLYEEVATYRLRTNNRSPQQVVAAVLHHLEID

SEQ ID NO: 7 - protein, 174 aa, Escherichia coli
MTQPLFLIGPRGCGKTTVGMALADSLNRRFVDTDQWLQSQLNMTVAEIVEREEWAGFRARETAALEAVTAPSTVIATGGGIILTEFNRHFMQNNGIVVYLCAPVSVLVNRLQAAPEEDLRPTLTGKPLSEEVQEVLEERDALYREVAHIIIDATNEPSQVISEIRSALAQTINC

SEQ ID NO: 8 - DNA, 179 bp, synthetic construct
  misc_feature 1..179: note = Pomotorsequenz
  source 1..179: mol_type = other DNA
cacagggtagctggtagtttgaaaatcaacgccgttgcccttaggattcagtaactggcacattttgtaatgcgctagatctgtgtgctcagtcttccaggctgcttatcacagtgaaagcaaaaccaattcgtggctgcgaaagtcgtagccaccacgaagtccaggaggacatacat

SEQ ID NO: 9 - protein, 430 aa, Escherichia coli
MVFVSDSSISLPIWDAPRARGPIVSDLAIPGSKSITNRALILAALASTPSTIIDVLRSRDTDLMTDGLRSLGITITEEAVDRYRVEPGQLSAGSVECGLAGTVMRFLPPVAAFADGPVHFDGDPQARVRPMTSILDALRSLGVEVDNNNLPFTVNAGEVPEGGVVEIDASGSSQFVSGLLLSAPRFKNGVTVKHVGGRLPSMPHIEMTVDMLRSAGIEIEESENQWVVHPGEILGRTWRIEPDLSNATPFLAAAAVTGGTIKINHWPIKTTQPGDAIRSILERMGCEVELVAQGEGYDLSVTGPVALKGIEIDMSDIGELTPTVAALAALASTESRLTGIAHLRGHETDRLAALTAEINKLGGKCTELKDGLLIEPASLHGGVWHSYADHRMATAGAIIGLAVDGVQVEDIKTTSKTFPGFENVWEEMVG

SEQ ID NO: 10 - protein, 410 aa, Corynebacterium glutamicum
MLGMLRWTTAGESHGQALIATVEHMPAGVPVTKDEVSYQLARRRLGYGRGARMKFEQDALTFLTGIRHGLTLGSPISIMIGNTEWDKWTTIMSSDALDMEDPDNVAAMSSGRGAKLTRPRPGHADYAGMLKYGFDDARNVLERSSARETAARVAAATVARSFLRETLGVEVLSHVISIGASEPYTGAEPTFADIQAIDDSPVRAFGKDAEESMIAEIEAAKKAGDTLGGIVEVIVEGLPIGLGSHISGEDRLDAQIAAALMGIQAIKGVEIGDGFEEARRRGSEAHDEVFLDDNGVYRNTNRAGGLEGGMTNGETLRVRAGMKPISTVPRALKTIDMENGKAATGIHQRSDVCAVPAAGVVAEAMVTLVLARAVLQKFGGDSLSETKSNIDTYLKNIEERMKFEGLEDGA

SEQ ID NO: 11 - protein, 350 aa, synthetic construct
  REGION 1..350: note = modifiziertes Enzym
MNYQNDDLRIKEIKELLPPVALLEKFPATENAANTVAHARKAIHKILKGNDDRLLVVIGPCSIHDPVAAKEYATRVLALREELKDELEIVMRVYFEKPRTTVGWKGLINDPHMDNSFQINDGLRIARKLLLDINDSGLPAAGEFLDMITPQYLADLMSWGAIGARTTESQVHRELASGLSCPVGFKNGTDGTIKVAIDAINAAGAPHCFLFVTKWGHSAIVNTSGNGDCHIILRGGKEPNYSAKHVAEVKEGLNKAGLPAQVMIDFSHANSSKQFKKQMDVCADVCQQIAGGEKAIIGVMVESHLVEGNQSLESGEPLAYGKSITDACIGWEDTDALLRQLANAVKARRG

SEQ ID NO: 12 - protein, 446 aa, Xanthomonas campestris
MSNTVFIGAKEYFPGIGKIGFEGRDSDNPLAFKVYDANKQVAGKTMAEHLRFAVAYWHSFCGNGADPFGPGTRAYPWDVGNTALARAEAKSDAAFEFFTKLGVPYYCFHDIDLAPDADDIGEYENNLKHMVGIAKQRQADTGVKLLWGTANLFSHPRYMNGASTNPDFNVVARAAVQVKAAIDATVELGGENYVFWGGREGYACLHNTQMKREQDNMARFLTLARDYGRAIGFTGNFLIEPKPMEPMKHQYDFDSATVIGFLRQHGLDQDFKLNIEANHATLSGHSFEHDLQVASDAGLLGSIDANRGNPQNGWDTDQFPTDLYDTVGAMLVVLRQGGLAPGGLNFDAKVRRESSDPQDLFLAHIGGMDAFARGLEVANALLTSSPLETWRAQRYASFDSGAGADFANGTSTLADLAKYAAGKGEPTQVSGRQEAYENLINQYLTR

SEQ ID NO: 13 - protein, 463 aa, synthetic construct
  REGION 1..463: note = modifiziertes Enzym
MSNTVFIGAKEYFPGIGKIGFEGRDSDNPLAFKVYDANKQVAGKTMAEHLRFAVAYWHSFCGNGADPFGPGTRAYPWDVGNTALARAEAKSDAAFEFFTKLGVPYYCFHDIDLAPDADDIGEYENNLKHMVGIAKQRQADTGVKLLWGTANLFSHPRYMNGASTNPDFNVVARAAVQVKAAIDATVELGGENYVFWGGREGYACLHNTQMKREQDNMARFLTLARDYGRAIGFTGNFLIEPKPMEPMKHQYDFDSATVIGFLRQHGLDQDFKLNIEANHATLSGHSFEHDLQVASDAGLLGSIDANRGNPQNGWDTDQFPTDLYDTVGAMLVVLRQGGLAPGGLNFDAKVRRESSDPQDLFLAHIGGMDAFARGLEVANALLTSSPLETWRAQRYASFDSGAGADFANGTSTLADLAKYAAGKGEPTQVSGRQEAYENLINQYLTRHRVAGSLKINAVALRIQ

SEQ ID NO: 14 - protein, 460 aa, Corynebacterium glutamicum
MALVLGIDSSTQSCKALLVDAATGQVIDEGRASHPSGSEVDPRAWIAALDQATEGLLERADAVSIAGQQHGMVALDENDEIVRPALLWNDTRSAQAALDLNEEIGGDQAAVDATGSVYVASLTATKMRWMRDHEPENAARTASVMLPHDFLTWHLMGRGRKVTDHGDASGTGYYSTRDRAWRTDLAALALGHEVELPELLAPNAIAGTTPGGVKVAAGTGDNAAAALGLDLQPGDVSVSIGTSGVAGMTVQHSVHDPSGLVTGFADATGAYFPLACTLNGAPVLEFGRRILGVEWEEFDALALAAQPGSGGVTLQPYLEGERTPNRPAARGVLAGLNCATTREDFARATVEGLLLALDDAVTALVEATGVPVQRIQLIGGGARSQAVREIAPEIFGHEIVVPEPAEYVALGAARQAAWALSGEATPPQWPTPGSDPHRAPKNTELSTRYAKLRAATQGWY

SEQ ID NO: 15 - DNA, 3302 bp, synthetic construct
  misc_feature 1..3302: note = Expressionskonstrukt
  source 1..3302: mol_type = other DNA
gatcccacagggtagctggtagtttgaaaatcaacgccgttgcccttaggattcagtaactggcacattttgtaatgcgctagatctgtgtgctcagtcttccaggctgcttatcacagtgaaagcaaaaccaattcgtggctgcgaaagtcgtagccaccacgaagtccaggaggacatacagtatgacacaacctctttttctgatcgggcctcggggctgtggtaaaacaacggtcggaatggcccttgccgattcgcttaaccgtcggtttgtcgataccgatcagtggttgcaatcacagctcaatatgacggtcgcggagatcgtcgaaagggaagagtgggcgggatttcgcgccagagaaacggcggcgctggaagcggtaactgcgccatccaccgttatcgctacaggcggcggcattattctgacggaatttaatcgtcacttcatgcaaaataacgggatcgtggtttatttgtgtgcgccagtatcagtcctggttaaccgactgcaagctgcaccggaagaagatttacggccaaccttaacgggaaaaccgctgagcgaagaagttcaggaagtgctggaagaacgcgatgcgctatatcgcgaagttgcgcatattatcatcgacgcaacaaacgaacccagccaggtgatttctgaaattcgcagcgccctggcacagacgatcaattgttgacaatctttagaggagacacaacatggtctttgtgtctgattcgtctatctctttgcccatttgggatgctccgcgcgctcgcggccccatagtctcggacctggctatccctggttccaagtcgatcaccaaccgcgccctcatcttggctgcgctcgcatcaactccatccaccatcattgatgtccttcgtagtcgtgataccgatctcatgactgatggtctacgcagcctcggaatcaccattactgaagaggcagtcgatcgctaccgcgttgagcccggacagttgtctgctggctccgttgagtgtggtcttgctggtacggtcatgcgctttttgcctcctgttgctgctttcgctgatggtcctgttcattttgatggcgatcctcaagctcgtgttcgtccgatgaccagcattttggatgcgctgcgttcgcttggtgtggaggtagacaacaacaatctgcctttcactgttaatgctggtgaggtccctgagggtggcgtggttgagattgatgcttccggctcatctcagtttgtttctggtcttttgctttcagcgcctcgttttaaaaatggcgtcaccgttaagcacgtcggtggtcgtctgccgagcatgccgcatattgagatgaccgtcgatatgcttcgttccgcaggcattgagatcgaagagtcagaaaatcagtgggttgttcatcctggtgagatcttgggtcggacctggcgcattgagccggatctttctaatgcgactccgttcctagctgccgctgcggtcactggtggaaccatcaagattaaccactggccaatcaaaactactcagcctggcgatgctattcgttcgattcttgagcgcatgggctgcgaagttgagctggttgctcagggtgaaggttacgatctgtcggtgactggtccggttgctctcaagggcattgagatcgatatgtccgatatcggtgagttgacccctaccgtggcggcgttggctgcgttggcgtcgacagagtctcgtttgaccggtattgctcatcttcgtggccatgagacggatcgtttggctgcgttgactgcggagatcaacaaacttggtggaaagtgcactgagcttaaggatggtctgttgattgagcctgcgtcgctgcacggtggtgtgtggcattcatatgctgatcaccgtatggctactgctggtgcgatcattggcctcgcggttgatggcgttcaggttgaagacattaagaccacttccaaaactttccctggttttgaaaatgtttgggaggagatggttggctaggcgaaagtcgtagccaccacgaagtccgaaaggaggcccttcagatgctaggcatgcttcgatggactacagcaggtgaatcccacggccaggcgcttatcgccacggttgaacacatgccagcaggcgtgcccgtgactaaagatgaggtctcgtatcaattggcgcgccgacgccttggatatggtcgcggcgctcgcatgaagtttgagcaagacgcgttgaccttcctcaccggcatccgccacggcctcactttgggtagccccatctcaatcatgatcggcaacactgagtgggataagtggaccaccatcatgtcctctgacgctttggacatggaagacccagataacgttgcggcgatgtcttcgggtcggggcgcaaaactgactcgtccgcgtccaggccacgctgattacgcaggcatgctcaagtacggattcgatgatgcccgcaacgtgctggagcgttcttcagcccgtgagacggcagcacgcgtggcagcagcaaccgttgcgcgttccttcctgcgtgaaaccttgggcgtggaagtgctttcccacgtaatttccattggtgcgtccgagccttacactggcgcggagccaacctttgcagatattcaagcaatcgatgattccccagttcgtgcattcggtaaagacgctgaagaatccatgatcgcggaaatcgaggccgcaaagaaagccggcgataccctcggtggcatcgtggaagtgattgttgaaggcctgcccatcggtttgggctcacacatttctggcgaagatcgcctcgatgcgcagatcgcagctgcactcatgggcattcaggccatcaagggcgtggaaatcggtgacggtttcgaagaagctcgtcgacgtggctccgaagcccacgatgaagtgttcctggatgacaacggcgtataccgcaacaccaaccgtgcaggtggcctcgaaggcggcatgaccaacggtgaaaccctgcgcgttcgtgctggcatgaagccaatttctactgtgcctcgcgccctgaaaaccattgatatggaaaacggcaaggcagcaaccggaatccaccagcgttccgacgtgtgcgctgttccagccgccggtgtcgttgcagaagcaatggtcaccctggttctcgcccgcgcagtcctgcagaaattcggcggtgactccctgagcgaaaccaagagcaacattgacacctacctcaaaaacattgaggaacgaatgaaattcgaaggtttagaggatggagcgtaa

SEQ ID NO: 16 - DNA, 3539 bp, synthetic construct
  misc_feature 1..3539: note = Expressionskonstrukt
  source 1..3539: mol_type = other DNA
cacagggtagctggtagtttgaaaatcaacgccgttgcccttaggattcagtaactggcacattttgtaatgcgctagatctgtgtgctcagtcttccaggctgcttatcacagtgaaagcaaaaccaattcgtggctgcgaaagtcgtagccaccacgaagtccaggaggacatacatgaaaggaggcccttcagatgtccaacaccgtgttcatcggcgcaaaggaatacttcccaggcatcggcaagatcggcttcgaaggccgcgattccgataacccactggcattcaaggtgtacgatgcaaacaagcaggtggcaggcaagaccatggcagaacacctgcgcttcgcagtggcatactggcactccttctgcggcaacggcgcagatccattcggcccaggcacccgcgcatacccatgggatgtgggcaacaccgcactggcacgcgcagaagcaaagtccgatgcagcattcgaattcttcaccaagctgggcgtgccatactactgcttccacgatatcgatctggcaccagatgcagatgatatcggcgaatacgaaaacaacctgaagcacatggtgggcatcgcaaagcagcgccaggcagataccggcgtgaagctgctgtggggcaccgcaaacctgttctcccacccacgctacatgaacggcgcatccaccaacccagatttcaacgtggtggcacgcgcagcagtgcaggtgaaggcagcaatcgatgcaaccgtggaactgggcggcgaaaactacgtgttctggggcggccgcgaaggctacgcatgcctgcacaacacccagatgaagcgcgaacaggataacatggcacgcttcctgaccctggcacgcgattacggccgcgcaatcggcttcaccggcaacttcctgatcgaaccaaagccaatggaaccaatgaagcaccagtacgatttcgattccgcaaccgtgatcggcttcctgcgccagcacggcctggatcaggatttcaagctgaacatcgaagcaaaccacgcaaccctgtccggccactccttcgaacacgatctgcaggtggcatccgatgcaggcctgctgggctccatcgatgcaaaccgcggcaacccacagaacggctgggataccgatcagttcccaaccgatctgtacgataccgtgggcgcaatgctggtggtgctgcgccagggcggcctggcaccaggcggcctgaacttcgatgcaaaggtgcgccgcgaatcctccgatccacaggatctgttcctggcacacatcggcggcatggatgcattcgcacgcggcctggaagtggcaaacgcactgctgacctcctccccactggaaacctggcgcgcacagcgctacgcatccttcgattccggcgcaggcgcagatttcgcaaacggcacctccaccctggcagatctggcaaagtacgcagcaggcaagggcgaaccaacccaggtgtccggccgccaggaagcatacgaaaacctgatcaaccagtacctgacccgccacagggtagctggtagtttgaaaatcaacgccgttgcccttaggattcagtaactggcacattttgtaatgcgctagatctgtgtgctcagtcttccaggctgcttatcacagtgaaagcaaaaccaattcgtggctgcgaaagtcgtagccaccacgaagtccaggaggacatacatgaaaggaggcccttcagatggctttggttcttggaatcgatagttccacccaatcctgcaaggctttgcttgtcgatgccgccaccggccaggttatcgacgaaggccgcgcgagtcacccgagcgggtcggaggtagatccacgtgcgtggatcgctgcgctggatcaagctaccgaggggttgttagaacgcgcggacgctgtatctattgcaggccagcagcacggcatggtggcgttggatgaaaacgatgaaatcgttcgcccggcgttgttatggaatgacactcgttctgcccaggctgcgttggatctcaatgaggagatcggcggcgatcaggctgcggtagatgccacgggaagtgtgtatgttgcttctttaactgccaccaaaatgcggtggatgcgtgatcatgaaccagaaaatgcagcgcgcacggcgtcggtgatgttgcctcatgatttcctcacctggcatttgatgggacgcggacgcaaagtcaccgaccatggtgatgcttctggaacgggctactacagcacgcgtgatcgtgcgtggcgcaccgatctagctgccttggcgctgggccatgaggtggaacttcctgaactcctggccccaaatgcgattgcgggaacaactccaggtggagtgaaagttgctgcaggcacgggagataatgctgcggctgcgcttggccttgatttgcagcctggtgatgtcagcgtgtcgatcggcacctctggcgttgccggcatgaccgttcaacatagcgtccacgatccatctggtttggtcactggtttcgccgatgccacgggtgcgtatttcccgctggcctgcacgcttaatggcgcaccggtgttggaattcggccgccgcattctgggcgtggaatgggaagagttcgatgcgcttgcactggctgctcaacccggttcaggtggcgtgacgctccagccttatttggagggcgagcgtacgccgaatcgtcccgcagcacgtggcgttttggctggactaaactgtgcaacgacccgcgaggactttgcccgagcaactgttgaaggcttgttgttggcattggatgatgctgtaacggcgctggttgaggccacgggagtgcccgttcagcgcatccagctcatcggtggcggcgcgcgttcacaggcggttcgtgagattgcccctgagattttcggccatgagattgtggttccagaacccgctgaatatgtggcgttgggtgcagctcgtcaggcggcatgggcgctgtcgggtgaggccacgccaccgcagtggccaactcccggttccgatccgcaccgcgcacctaaaaacactgagctgagcacgcgttatgcgaagctgcgtgctgcaacgcagggttggtactagggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactct

SEQ ID NO: 17 - DNA, 1101 bp, Corynebacterium glutamicum
  source 1..1101: mol_type = other DNA
atgagttctccagtctcactcgaaaacgcggcgtcaaccagcaacaagcgcgtcgtggctttccacgagctgcctagccctacagatctcatcgccgcaaacccactgacaccaaagcaggcttccaaggtggagcaggatcgccaggacatcgctgatatcttcgctggcgacgatgaccgcctcgttgtcgttgtgggaccttgctcagttcacgatcctgaagcagccatcgattacgcaaaccgcctggctccgctggcaaagcgccttgatcaggacctcaagattgtcatgcgcgtgtacttcgagaagcctcgcaccatcgtcggatggaagggattgatcaatgatcctcacctcaacgaaacctacgacatcccagagggcttgcgcattgcgcgcaaagtgcttatcgacgttgtgaaccttgatctcccagtcggctgcgaattcctcgaaccaaacagccctcagtactacgccgacactgtcgcatggggagcaatcggcgctcgtaccaccgaatctcaggtgcaccgccagctggcttctgggatgtctatgccaattggtttcaagaacggaactgacggaaacatccaggttgcagtcgacgcggtacaggctgcccagaacccacacttcttcttcggaacctccgacgacggcgcgctgagcgtcgtggagaccgcaggcaacagcaactcccacatcattttgcgcggcggtacctccggcccgaatcatgatgcagcttcggtggaggccgtcgtcgagaagcttggtgaaaacgctcgtctcatgatcgatgcttcccatgctaactccggcaaggatcatatccgacaggttgaggttgttcgtgaaatcgcagagcagatttctggcggttctgaagctgtggctggaatcatgattgagtccttcctcgttggtggcgcacagaaccttgatcctgcgaaattgcgcatcaatggcggtgaaggcctggtgtacggacagtctgtgaccgataagtgcatcgatattgacaccaccatcgatttgctcgctgagctggccgcagcagtaagggaacgccgagcagcagccaagtaa

SEQ ID NO: 18 - protein, 366 aa, synthetic construct
  REGION 1..366: note = Modifiziertes Enzym
MSSPVSLENAASTSNKRVVAFHELPSPTDLIAANPLTPKQASKVEQDRQDIADIFAGDDDRLVVVVGPCSVHDPEAAIDYANRLAPLAKRLDQDLKIVMRVYFEKPRTIVGWKGLINDPHLNETYDIPEGLRIARKVLIDVVNLDLPVGCEFLNPNSPQYYADTVAWGAIGARTTESQVHRQLASGMSMPIGFKNGTDGNIQVAVDAVQAAQNPHFFFGTSDDGALSVVETAGNSNSHIILRGGTSGPNHDAASVEAVVEKLGENARLMIDASHANSGKDHIRQVEVVREIAEQISGGSEAVAGIMIESFLVGGAQNLDPAKLRINGGEGLVYGQSVTDKCIDIDTTIDLLAELAAAVRERRAAAK

SEQ ID NO: 19 - DNA, 1053 bp, synthetic construct
  source 1..1053: mol_type = other DNA
atgaattatcagaacgacgacctgcgcatcaaagaaattaaagaactgctgcctccggttgcactgctggaaaaatttccggcaaccgaaaatgcagcaaataccgttgcacatgcacgtaaagccattcacaaaattctgaaaggtaatgatgatcgcctgctggttgttattggtccgtgtagcattcatgatccggttgccgcaaaagaatatgcaacccgtctgctggcactgcgtgaagaactgaaagatgaactggaaattgtgatgcgcgtgtattttgaaaaaccgcgtaccaccgttggttggaaaggtctgattaatgatccgcacatggataacagctttcagattaacgatggtctgcgtattgcccgtaaactgctgctggatattaatgatagcggtctgcctgcagccggtgaatttctgaatatgattacaccgcagtatctggccgatctgatgagctggggtgcaattggtgcacgcaccaccgaaagccaggttcatcgtgaactggcaagcggtctgagctgtccggttggctttaaaaacggcaccgatggcaccattaaagttgcaattgatgcaattaatgcagcgggtgcaccgcattgttttctgagcgttaccaaatggggtcatagcgcaattgttaataccagcggtaatggtgattgccatattattctgcgtggtggtaaagaaccgaattattcagcaaaacatgtggccgaagttaaagaaggtctgaataaagcaggtctgccagcacaggttatgattgattttagccatgcaaatagcagcaaacagttcaaaaaacaaatggatgtttgtgccgatgtgtgtcagcagattgccggtggtgaaaaagcaattattggtgttatggttgaaagccatctggtggaaggtaatcagagcctggaaagcggtgaaccgctggcctatggtaaaagcattaccgatgcatgtattggttgggaagataccgatgccctgctgcgtcagctggcaaatgcagtaaaagcgcgtcgcgggtaa

SEQ ID NO: 20 - DNA, 1053 bp, synthetic construct
  source 1..1053: mol_type = other DNA
atgaactaccagaacgatgatctgcgcatcaaagagatcaaagaactgctgccaccagtggcactgctggaaaagttcccagcaaccgaaaacgcagcaaacaccgtggcacacgcacgcaaggcaatccacaagatcctgaagggcaacgatgaccgcctgctcgtggtgatcggtccatgctccatccacgatccagtggcagcaaaagaatacgcaacccgcttgctggcactgcgcgaagaactgaaggatgaactggaaatcgtgatgcgcgtgtacttcgaaaagccacgcaccaccgtcggctggaagggcctgatcaacgatccacacatggataattccttccagatcaacgacggcctgcgcattgcacgcaagctgctgctggacatcaacgattccggtctgccagccgcaggcgagttcctggatatgatcaccccacagtacctggccgatctgatgtcctggggtgcaatcggtgctcgcaccactgaatcccaggtgcaccgcgaactggcatccggtctgttctgcccagtgggcttcaagaacggcaccgacggcaccatcaaggtggcaatcgatgcaatcaacgcagcaggcgcaccacactgctttctgtccgtgaccaagtggggtcactccgcaatcgtgaacacctccggcaacggcgactgccacatcatccttcgcggtggcaaagaaccaaactactccgcaaagcacgtggcagaagtgaaagaaggcctgaacaaggctggcctgccagcacaggtgatgatcgatttctcccacgcaaactcctccaagcagttcaaaaagcagatggatgtgtgcgcagatgtgtgccagcagatcgcaggcggtgaaaaggcaatcatcggcgtgatggtggaatcccacctggtggaaggcaaccagtccttggaatccggtgaaccactggcatacggcaagtccatcaccgatgcctgcatcggctgggaagataccgatgcactgctgcgccagctcgcaaacgcagtgaaggcacgccgtggctaa

(19) United States
(12) Patent Application Publication
(10) Pub. No.: US 2025/0043254 A1
(43) Pub. Date: Feb. 6, 2025

(54) PARTICULARLY SUITABLE 3-DESOXYARABINOHEPTULOSANATE-7-PHOSPHATE SYNTHASE FOR THE FERMENTATIVE PREPARATION OF ORTHO-AMINOBENZOIC ACID

(71) Applicant: Covestro Deutschland AG, Leverkusen (DE)

(72) Inventors: Simon Klaffl, Düsseldorf (DE); Georg Sprenger, Backnang (DE); Katrin Gottlieb, Stuttgart (DE); Jung-Won Youn, Stuttgart (DE)

(21) Appl. No.: 18/718,579

(22) PCT Filed: Dec. 14, 2022
(86) PCT No.: PCT/EP2022/085919

§ 371 Date: Jun. 11, 2024

(30) Foreign Application Priority Data
Dec. 17, 2021 (EP) 21215753.1

(51) Int. Cl. C12N 9/10; C12P 13/00

(52) U.S. Cl. CPC C12N 9/1085; C12P 13/005; C12Y 205/01054

(57) ABSTRACT

The present invention relates to the use of specific variants of 3-deoxyarabinoheptulosanate 7-phosphate synthase for producing ortho-aminobenzoic acid by means of microbial fermentation and microorganisms suitable for this purpose.

The present invention relates to the use of specific variants of 3-deoxyarabinoheptulosanate 7-phosphate synthase for producing ortho-aminobenzoic acid by means of microbial fermentation and microorganisms suitable for this purpose.

The production of ortho-aminobenzoic acid (oAB) with the aid of a series of genetically modified microorganisms is known from the prior art, for example from Balderas-Hernandez et al. (2009) “Metabolic engineering for improving anthranilate synthesis from glucose in Escherichia coli”, WO 2015/124687 and WO 2017/102853.

A variant of 3-deoxyarabinoheptulosanate 7-phosphate synthase is known from EP 0 077 196, the activity of which is less strongly inhibited in the presence of aromatic amino acids than is the case in other variants of this enzyme. Use of this enzyme in Corynebacterium glutamicum has not been demonstrated. It has also not been demonstrated that the production of oAB by fermentation can be improved by using this gene.

EP 2 147 972 discloses variants of DAHP synthase that deviate from the amino acid sequence of DAHP synthase as defined in SEQ ID NO. 11. This also applies to WO 2018/203947. WO 2019/073033, WO 2020/270587 and Bongaerts et al., 2001, “Metabolic engineering for microbial production of aromatic amino acids”, Metabolic Engineering, 3: 289-300.

In the study on which the present patent application is based, it was surprisingly found that Corynebacterium glutamicum produces more oAB in the presence of the DAHP synthase variant described in EP 0 077 196 than is the case with the use of other enzyme variants. This invention and advantageous embodiments thereof are defined in more detail in the patent claims and subsequently in the description.

The present invention relates in a first embodiment to the use of a 3-deoxyarabinoheptulosanate 7-phosphate synthase (DAHP synthase) having an amino acid sequence as defined by SEQ ID NO. 11 or a variant thereof for producing ortho-aminobenzoic acid (oAB) by microbial fermentation. When the present application refers to “production” with respect to oAB or another compound of the shikimate pathway, it refers to a process in the course of which the compound in question is released from the cell into the surrounding medium in industrially relevant amounts and accumulates therein. In the course of this process of release and enrichment, extracellular concentrations of the relevant compound of at least 1 g/L, more preferably at least 2 g/L and most preferably at least 4 g/L are preferably achieved. The mere existence of a compound as an intracellular intermediate of a metabolic pathway is not “production” in the context of the present patent application.

### Use

The “use” of 3-deoxyarabinoheptulosanate 7-phosphate synthase (DAHP synthase) having an amino acid sequence as defined by SEQ ID NO. 11 consists in that said enzyme is recombinantly expressed in at least one microbial cell, and that this at least one microbial cell recombinantly expressing said enzyme is cultured under conditions that enable metabolic activity of the cell.

The use according to the invention of the DAHP synthase as defined by SEQ ID NO. 11 does not exclude that the microbial cell additionally comprises further genetic modifications, in particular expresses further proteins recombinantly. These modifications are preferably those disclosed below in this application.

Since the improved 3-deoxyarabinoheptulosanate 7-phosphate synthase increases the flow of metabolites through the entire shikimate pathway to oAB, the improved DAHP synthase according to the invention in a preferred embodiment of the present invention is used for the synthesis of at least one compound selected from the group consisting of 3-deoxyarabinoheptulosanate 7-phosphate, 3-dehydroquinate, 3-dehydroshikimate, shikimate, shikimate 3-phosphate, chorismate, prephenate and ortho-aminobenzoic acid by means of microbial fermentation. In a further preferred embodiment of the present invention, the improved 3-deoxyarabinoheptulosanate 7-phosphate synthase is used to produce at least one compound selected from the group consisting of methylanthranilic acid, para-aminobenzoic acid, vanillin, indole, indoxyl, indigo, mandelic acid, ferulic acid and cinnamic acid. Particular preference is given to the use for producing oAB, methylanthranilic acid or para-aminobenzoic acid.

All definitions given below in this application also apply to this embodiment. The person skilled in the art understands that the metabolic and genetic configuration of the microbial cells or cells used must be adapted to the desired product by reducing or completely preventing the activity of enzymes that catalyze the conversion of the desired product to secondary products.

3-Deoxyarabinoheptulosanate 7-phosphate synthase

The term “3-deoxyarabinoheptulosanate 7-phosphate synthase” refers to the enzyme that catalyzes the first reaction of the shikimic acid pathway, namely the reaction of erythrose 4-phosphate and phosphoenolpyruvate to give 3-deoxyarabinoheptulosanate 7-phosphate and an inorganic phosphate. The enzyme variant having an amino acid sequence according to SEQ ID NO. 11 was disclosed in EP 0 077 196.

A “variant” of the DAHP synthase as defined in SEQ ID NO. 11 is an enzyme obtained by adding, deleting or exchanging up to 5%, preferably up to 2%, of the amino acids present in the respective enzyme, with the proviso that positions 76 and 211 remain unaltered. It is also preferred that in addition positions 10, 13, 147, 148, 150, 151, 179, 209, 211 and 212 also remain unaltered. In a more preferred embodiment of the present invention, additionally positions 144, 175 and 215 are also unaltered in the variant. In an even more preferred embodiment of the present invention, also positions 92, 97, 165, 186 and 268 are unaltered in addition to the aforementioned positions. The skilled person understands that the aforementioned positions shift accordingly if amino acids are deleted or inserted. The aforementioned modifications may in principle be executed continuously or discontinuously at any desired point in the enzyme. However, they are preferably executed only at the N-terminus and/or at the C-terminus of the polypeptide. Amino acid substitutions are preferably conservative substitutions, i.e. those in which the modified amino acid has a residue with similar chemical properties to the amino acid present in the unaltered enzyme. Amino acids having basic residues are therefore particularly preferably exchanged for those with basic residues, amino acids having acidic residues for those with likewise acidic residues, amino acids having polar residues for those with polar residues and amino acids having non-polar residues for those with non-polar residues. It is particularly preferred that the variant of DAHP synthase also possesses an appropriate enzyme activity. The specific enzyme activity of the variant is particularly preferably at least 80% of the specific activity of the unmodified DAHP synthase according to SEQ ID NO. 11.

### Ortho-Aminobenzoic Acid

The term oAB refers to ortho-aminobenzoic acid, also known by the name “anthranilic acid”. Since this compound has both a carboxyl group having acidic properties and an amino group having basic properties, the charge of the molecule depends on pH. In the context of the present application, the term “ortho-aminobenzoic acid” refers to the molecule irrespective of whether it has a positive, a negative or no net charge.

### Microbial Fermentation

The term “microbial fermentation” refers to the incubation of one or more microbial cells in a suitable culture medium under conditions that enable metabolic activity of the cells. In particular, this means setting and maintaining a suitable pH, a suitable oxygen saturation, a suitable temperature and a suitable osmolarity. The person skilled in the art may select culture conditions that are suitable for the microorganism in question based on their expertise and the generally available literature. Corynebacterium, in particular Corynebacterium glutamicum, is preferably cultured at a pH between 6 and 8, at a temperature between 25° C. and 40° C. and at an osmolality between 400 and 2600 mOsmol/kg. The oxygen saturation is preferably kept as close as possible to the maximum achievable under standard pressure and standard atmosphere.

In particular, at least one fermentable carbon source is also provided to the microbial cells during microbial fermentation. This is an organic carbon compound that can be utilized by the cells to form oAB. It is preferred that it can additionally also be used as an energy source for metabolism and as a carbon source for the development of microbial biomass. In one embodiment of the present invention, the metabolic pathways of the microbial cell for the formation of microbial biomass and the formation of oAB are separated to such an extent that at least one fermentable carbon source is used for energy metabolism and constructive metabolism and at least one other is used for product formation. Preferred fermentable carbon sources are selected from the group consisting of C-5 monosaccharides, C-6 monosaccharides, disaccharides and trisaccharides. Preferred C-5 monosaccharides are xylose and arabinose. Preferred C-6 monosaccharides are glucose, fructose and mannose. A preferred disaccharide is sucrose. A preferred trisaccharide is kestose.

The metabolic activity preferably consists in the production of oAB and/or the construction of biomass, particularly preferably in the production of oAB. A suitable parameter that allows continuous monitoring of metabolic activity for process control is also the rate of oxygen uptake. This may be carried out continuously, for example by oxygen electrodes, and provides measurement results without delay, which can be used to adjust the culture conditions.

### Culture Medium

Culture media suitable for culturing microbial cells, especially Corynebacterium sp., are known from the prior art. Suitable culture media contain at least one buffer to regulate the pH, sources of inorganic nutrients that can be utilized by the microorganism used, in particular nitrogen, sulfur and phosphorus, and also the trace elements required by the organism used. Depending on the microorganism, the addition of vitamins and/or complex media constituents such as peptone or yeast extract may be useful. Particularly suitable culture media are described in the working examples included in this application.

### Microbial Cell

The microbial cell is preferably a bacterial cell, more preferably a strain of the genus Corynebacterium and even more preferably Corynebacterium glutamicum. When the term “a microbial cell” is used in the context of this application, a plurality of cells is always included, since the formation of an appreciable amount of product within a reasonable period of time requires the presence of a large number of cells. In addition, the culturing of a cell usually also leads to cell multiplication, so that even in the presence of a single cell in the culture medium, a plurality of cells is formed after some time.

The microbial cell is a recombinant microbial cell. This means that this cell comprises at least one nucleic acid sequence that codes for a gene that is not endogenously present in the cell in question (“foreign gene”). This foreign gene may also be a variant of an endogenously occurring enzyme, which differs from the endogenously occurring enzyme only by a higher or lower enzyme activity or which is subject to a different regulatory mechanism. This foreign gene must be present in a form that allows the expression thereof. This means in particular that it is under control of a promoter suitable for the relevant microbial cell. In one embodiment of the present invention, the foreign gene with the promoter sequence suitable for the microorganism in question is present in a plasmid.

In another embodiment, the foreign gene with the promoter sequence suitable for the microorganism in question is integrated into the genome of the microbial cells. Mandatorily, the recombinant microbial cell comprises a gene coding for a DAHP synthase having a sequence as defined by SEQ ID NO: 11 or a variant thereof, with the proviso that positions 76 and 211 remain unaltered.

In a preferred embodiment of the present invention, the microbial cell is a cell which, in addition to the presence of the DAHP synthase according to SEQ ID NO. 11 defined above or a variant thereof, differs from the wild type of the microorganism in question at least by the following feature:

reduced activity of anthranilate phosphoribosyltransferase compared to the respective wild type, but where residual activity must be present. The residual activity is preferably between 10% and 60%, more preferably between 20% and 50% of the activity natively present in C. glutamicum ATCC13032. This is preferably achieved by reduced expression of the gene for the anthranilate phosphoribosyltransferase (trpD) compared to the wild type, although the expression is not completely suppressed. This is preferably effected by using a promoter sequence which has a lower transcription activity compared to the endogenous promoter sequence or by modifying the distance of the ribosome binding site to the start codon of the trpD gene or by altering the start codon itself. In a preferred embodiment of the present invention, the activity of the anthranilate phosphoribosyltransferase is reduced by deletion or inactivation of the gene for the endogenous anthranilate phosphoribosyltransferase (trpD) and the replacement of this gene by a gene for an anthranilate phosphoribosyltransferase having a modified ribosomal binding site and optionally a modified start codon as defined in SEQ ID NO. 1 or 2, preferably SEQ ID NO. 2. The amino acid sequence of the anthranilate phosphoribosyltransferase preferably corresponds to the endogenous anthranilate phosphoribosyltransferase, particularly preferably as defined by SEQ ID NO. 3 or a variant thereof.

In a more preferred embodiment, the microbial cell is additionally characterized (ii) by the deletion or inactivation of the gene coding for the phosphoenolpyruvate carboxylase as defined in SEQ ID NO. 4. This may be effected in any manner familiar to those skilled in the art, preferably by deleting the gene or a partial sequence thereof, by introducing at least one stop codon or by deleting or inactivating the promoter sequence. Particular preference is given to the deletion of at least part of the protein-coding sequence of the gene (SEQ ID NO. 5).

In an even more preferred embodiment of the present invention, the microbial cell is additionally characterized by at least one of the features cited below:
(iii) increased activity of shikimate kinase. This is preferably achieved by increased expression of a corresponding enzyme. In one embodiment of the present invention, the activity is increased by enhanced expression of the gene for the endogenous shikimate kinase as defined in SEQ ID NO. 6 or a variant thereof. In another preferred embodiment, this is achieved by expression of an exogenous shikimate kinase, preferably as defined in SEQ ID NO. 7 or a variant thereof. Enhanced expression of a gene can be achieved by any method known to those skilled in the art, in particular by introducing two or more copies of the corresponding gene into the microorganism or by using stronger promoters to express the endogenous enzyme. A particularly preferred promoter for the expression of foreign genes or the enhanced expression of endogenous genes is ptuf as defined in SEQ ID NO. 8.
(iv) enhanced activity of 3-phosphoshikimate 1-carboxyvinyltransferase and chorismate synthase. Preferably, these enzymes have an amino acid sequence as defined in SEQ ID NO 9 or a variant thereof and SEQ ID NO 10 or a variant thereof. This is preferably effected by introducing additional copies of the genes coding for these enzymes into the microorganism. The ptuf promoter is preferably used to control expression.

Based on anthranilate phosphoribosyltransferase (SEQ ID NO. 3), shikimate kinase (SEQ ID NO. 6 or 7), 3-phosphoshikimate 1-carboxyvinyltransferase (SEQ ID NO. 9) and chorismate synthase (SEQ ID NO. 10), “variant” signifies an enzyme obtained by adding, deleting or exchanging up to 10%, preferably up to 5%, of the amino acids present in the respective enzyme. The aforementioned modifications may in principle be executed continuously or discontinuously at any desired point in the enzyme. However, they are preferably executed only at the N-terminus and/or at the C-terminus of the polypeptide. Amino acid substitutions are preferably conservative substitutions, i.e. those in which the modified amino acid has a residue with similar chemical properties to the amino acid present in the unaltered enzyme. Amino acids having basic residues are therefore particularly preferably exchanged for those with basic residues, amino acids having acidic residues for those with acidic residues, amino acids having polar residues for those with polar residues and amino acids having non-polar residues for those with non-polar residues. Preferably, the specific enzyme activity of a variant is preferably at least 80% of the specific activity of the unmodified enzyme. Enzyme tests to verify the activity of the aforementioned enzymes can be found in the literature by those skilled in the art.

The study on which the present invention is based has demonstrated that 3-deoxyarabinoheptulosanate 7-phosphate synthase having an amino acid sequence as defined in SEQ ID NO. 11 improves the growth of production strains, substrate uptake, product formation, and also product yield.

In a further preferred embodiment, the present invention relates to a recombinant cell of the genus Corynebacterium, of the genus Bacillus or of the genus Pseudomonas comprising a gene coding for a DAHP synthase as defined in SEQ ID NO. 11 or a variant thereof. Preferably, said cell is a cell of a strain of Corynebacterium glutamicum, more preferably C. glutamicum ATCC13032. In the genus Bacillus, preference is given to Bacillus subtilis. In the genus Pseudomonas, preference is given to Pseudomonas putida.

In a preferred embodiment of the present invention, this cell comprises the following further genetic modification:

reduced activity of anthranilate phosphoribosyltransferase compared to the respective wild type, but where residual activity must be present. The residual activity is preferably between 10% and 60%, more preferably between 20% and 50% of the activity natively present in C. glutamicum ATCC13032. This is preferably achieved by reduced expression of the gene for the anthranilate phosphoribosyltransferase (trpD) compared to the wild type, although the expression is not completely suppressed. This is preferably effected by using a promoter sequence which has a lower transcription activity compared to the endogenous promoter sequence or by modifying the distance of the ribosome binding site to the start codon of the trpD gene or by altering the start codon itself. In a preferred embodiment of the present invention, the activity of the anthranilate phosphoribosyltransferase is reduced by deletion or inactivation of the gene for the endogenous anthranilate phosphoribosyltransferase (trpD) and the replacement of this gene by a gene for an anthranilate phosphoribosyltransferase having a modified ribosomal binding site and optionally a modified start codon as defined in SEQ ID NO. 1 or 2, preferably SEQ ID NO. 2. The amino acid sequence of the anthranilate phosphoribosyltransferase preferably corresponds to the endogenous anthranilate phosphoribosyltransferase, particularly preferably as defined by SEQ ID NO. 3 or a variant thereof.

In a more preferred embodiment of the present invention, the microbial cell is additionally characterized (ii) by the deletion or inactivation of the gene coding for the phosphoenolpyruvate carboxylase as defined in SEQ ID NO. 4. This may be effected in any manner familiar to those skilled in the art, preferably by deleting the gene or a partial sequence thereof, by introducing at least one stop codon or by deleting or inactivating the promoter sequence. Particular preference is given to the deletion of at least part of the protein-coding sequence of the gene (SEQ ID NO. 5).

In an even more preferred embodiment of the present invention, the microbial cell is additionally characterized by at least one of the features cited below:
(iii) increased activity of shikimate kinase. This is preferably achieved by increased expression of a corresponding enzyme. In one embodiment of the present invention, the activity is increased by enhanced expression of the gene for endogenous shikimate kinase as defined in SEQ ID NO. 6 or a variant thereof. In another preferred embodiment, this is achieved by expression of an exogenous shikimate kinase, preferably as defined in SEQ ID NO. 7 or a variant thereof. Enhanced expression of a gene can be achieved by any method known to those skilled in the art, in particular by introducing two or more copies of the corresponding gene into the microorganism or by using stronger promoters to express the endogenous enzyme present. A particularly preferred promoter for the expression of foreign genes or the enhanced expression of endogenous genes is ptuf as defined in SEQ ID NO. 8.
(iv) enhanced activity of 3-phosphoshikimate 1-carboxyvinyltransferase and chorismate synthase. Preferably, these enzymes have an amino acid sequence as defined in SEQ ID NO 9 or a variant thereof and SEQ ID NO 10 or a variant thereof. This is preferably effected by introducing additional copies of the genes coding for these enzymes into the microorganism. The ptuf promoter is preferably used to control expression.

All definitions given above, in particular for “variants” of the enzymes, also apply to this embodiment.

### Use of a Recombinant Microbial Cell

In yet another embodiment, the present invention relates to the use of a recombinant cell comprising a gene coding for a DAHP synthase as defined in SEQ ID NO. 11 or a variant thereof, with the proviso that positions 76 and 211 remain unaltered, for producing oAB by microbial fermentation.

All definitions given above in this application also apply to this embodiment. The “use” consists preferably in the culturing of the cell in a suitable culture medium in the presence of a fermentable carbon source which can be converted by this cell to oAB.

In a preferred embodiment of the present invention, the recombinant cell is used for the synthesis of at least one compound selected from the group consisting of 3-deoxyarabinoheptulosanate 7-phosphate, 3-dehydroquinate, 3-dehydroxyshikimate, shikimate, shikimate 3-phosphate, chorismate, prephenate and ortho-aminobenzoic acid by means of microbial fermentation.

The person skilled in the art understands that the metabolic and genetic configuration of the microbial cells or cells used must be adapted to the desired product by reducing or completely preventing the activity of enzymes that catalyze the conversion of the desired product to secondary products.

### Process

In a further embodiment, the present invention relates to a process comprising the steps of
a) providing an at least one recombinant microbial cell comprising a gene coding for a DAHP synthase as defined in SEQ ID NO. 11 or a variant thereof, with the proviso that positions 76 and 211 remain unaltered;
b) culturing the at least one recombinant microbial cell in a culture medium comprising at least one fermentable carbon source, wherein at least one compound is produced selected from the group consisting of oAB, 3-deoxyarabinoheptulosanate 7-phosphate, 3-dehydrocquinate, 3-dehydroshikimate, shikimate, shikimate 3-phosphate, chorismate, prephenate, methylanthranilic acid, para-aminobenzoic acid, vanillin, indole, indoxyl, indigo, mandelic acid, ferulic acid and cinnamic acid.

All definitions given above also apply to this embodiment. The recombinant microbial cells are “cultured” under the conditions described above in this application for “microbial fermentation”.

Since the process is aimed at the efficient production of oAB, the person skilled in the art understands that the phrase “at least one recombinant microbial cell” in this embodiment preferably signifies a plurality of cells, particularly preferably at least 10 cells.

The “provision” of the recombinant microbial cell signifies that, at the end of the process step, a viable recombinant cell is present as described above in this application.

The process step b) is preferably carried out for 15 to 80 hours. In a preferred embodiment, process step b) is carried out until a concentration of at least 1 g/L, preferably at least 6 g/L, and most preferably at least 9 g/L oAB is reached.

### Composition

In yet another embodiment, the present invention relates to a composition comprising
a) at least one microbial cell of the genus Corynebacterium comprising a gene coding for a DAHP synthase as defined in SEQ ID NO. 11 or a variant thereof, with the proviso that positions 76 and 211 remain unaltered;
b) at least one fermentable carbon source; and
c) at least one nitrogen source, at least one phosphorus source, at least one sulfur source and trace elements.

All definitions given above also apply to this embodiment. Preferably, the composition according to the invention is a culture medium comprising the recombinant microbial cell in viable form. The fermentable carbon source is one that can be utilized by the microbial cell for oAB production, particularly preferably glucose, xylose or a mixture thereof. The person skilled in the art understands that the term “at least one” in this embodiment preferably signifies a plurality of cells, particularly preferably at least 10⁹ cells, in order to ensure a sufficient space-time yield of the microbial fermentation.

In a preferred embodiment of the invention, the composition according to the invention additionally comprises oAB, more preferably at least 1 g/L, even more preferably at least 6 g/L and most preferably at least 9 g/L oAB.

The working examples which follow serve merely to illustrate the invention. They are not intended to limit the scope of protection of the claims in any way.

### EXAMPLES

### Material and Methods

The production strains and expression plasmids used are shown in Table 1. C. glutamicum strains were aerobically cultured at 30° C. in CGXII minimal medium (see Table 2) with 2% (w/v) glucose as carbon source and an optical density (OD) of 1. Pre-culturing was carried out in SY and then VKII medium (see Table 2). The main culturings were carried out either in 500 ml glass shaker flasks with 30 ml of medium on a rotary shaker at 200 rpm or in 250 ml glass bioreactors with 100 ml of medium in a fed-batch fermentation process. The gas supply and stirrer speed in the bioreactor was adjusted to a dissolved oxygen (DO) content of 30%, 20 g/L glucose were initially charged at the start of the fermentation. If the DO in the medium exceeded 37%, glucose was dissolved by a bolus addition to give a concentration of 20 g/L glucose. For pH control, a mixture of 1.5 mol/L NaOH and 4.9 mol/L (NH₄)₂OH was metered in.

To analyze the growth, OD measurements were carried out at a wavelength of 600 nm. In addition, the dry weight of the cells was determined. The supernatant of the samples was used to quantify glucose on a CedexBio Analyzer and in addition to measure the product concentration in the medium by HPLC analysis on a Luna 3 μm C18(2) separation column.

TABLE 1
Production strains and expression plasmids used for this patent

| Strain or plasmid | Description |
|---|---|
| Strain | |
| C. glutamicum Δppc | Derivative of ATCC13032 with functional |
| ΔtrpD::trpD5 | deletion of phosphoenolpyruvate carboxylase |
| | (SEQ ID NO. 4) and genomic exchange of trpD |
| | by SEQ ID NO. 1 |
| C. glutamicum Δppc | Derivative of ATCC13032 with functional |
| ΔtrpD::trpD5 ΔaroF | deletion of phosphoenolpyruvate carboxylase |
| | (SEQ ID NO. 4) and aroF and also genomic |
| | exchange of trpD by SEQ ID NO. 1 |
| Plasmid | |
| pEKEx2 | Kan^{R}; C. glutamicum/E. coli shuttle vector for |
| | regulatable gene expression (P_{tac} lacI^{Q} pBL1 |
| | oriV_{Cg} pUC18 oriV_{Ec}) |
| pEKEx2_aroF^{wt} | Kan^{R}; pEKEx2 derivative bearing the native aroF |
| | gene (SEQ ID NO. 17) from C. glutamicum |
| | under control of the tac promoter |
| pEKEx2_aroF^{E154N} | Kan^{R}; pEKEx2 derivative bearing a gene variant |
| | of the aroF gene from C. glutamicum with |
| | codon exchange for the amino acid exchange |
| | E154N (SEQ ID NO. 18) under control of the tac |
| | promoter |
| pEKEx2_aroG^{new} | Kan^{R}; pEKEx2 derivative bearing a gene variant |
| | of the aroG gene from E. coli with codon |
| | exchange for the amino acid exchange L76V |
| | and S211F (SEQ ID NO. 11) under control of the |
| | tac promoter |
| pEKEx2_aroG^{D146N} | Kan^{R}; pEKEx2 derivative bearing a gene variant |
| | of the aroG gene from E. coli with codon |
| | exchange for the amino acid exchange D146N |
| | (SEQ ID NO. 19) under control of the tac |
| | promoter. Codon-optimized for C. glutamicum |
| pEKEx2_aroG^{S180F} | Kan^{R}; pEKEx2 derivative bearing a gene variant |
| | of the aroG gene from E. coli with codon |
| | exchange for the amino acid exchange S180F |
| | (SEQ ID NO. 20) under control of the tac |
| | promoter. Codon-optimized for C. glutamicum |

TABLE 2
Composition of pre-culture and main culture media and also stock solutions
SY medium for pre-cultures of shaking flasks and fermentations

| Component | Concentration | | Comment |
|---|---|---|---|
| Soy peptone | 16 | g/l | — |
| Yeast extract | 10 | g/l | — |
| NaCl | 5 | g/l | — |

Adjust pH to 7.0 with KOH and autoclave

| | | | |
|---|---|---|---|
| Glucose*H2O | 15 | g/l | Addition after autoclaving |
| Kanamycin | 50 | μg/L | |

VKII medium for pre-cultures of shaking flasks and fermentations

| Component | Concentration | | Comment |
|---|---|---|---|
| KH₂PO₄ | 1 | g/l | — |
| K₂HPO₄ | 1 | g/l | — |
| (NH₄)₂SO₄ | 20 | g/l | — |
| CaCl₂*2H₂O | 0.01 | g/l | — |
| MgSO₄*7H₂O | 0.25 | g/l | — |
| MOPS | 42 | g/l | — |
| UREA | 5 | g/l | — |
| Yeast extract | 5 | g/l | — |

Adjust pH to 7.0 with KOH and autoclave

| | | | |
|---|---|---|---|
| Trace element stock solution (1000x) | 1 | mL/L | Addition after autoclaving |
| Biotin | 0.002 | g/l | — |
| Glucose*H2O | 40 | g/l | — |
| Kanamycin | 50 | μg/ml | |

CGXII Minimum medium for shaking flask cultures

| Component | Concentration | | Comment |
|---|---|---|---|
| KH₂PO₄ | 1 | g/l | — |
| K₂HPO₄ | 1 | g/l | — |
| (NH₄)₂SO₄ | 10 | g/l | — |
| MOPS | 62 | g/l | — |
| CaCl₂*2H₂O | 0.01 | g/l | — |
| MgSO₄*7H₂O | 0.25 | g/l | — |

Adjust pH to 7.0 with KOH and autoclave

| | | | |
|---|---|---|---|
| Trace element stock solution (1000x) | 1 | mL/L | Addition after autoclaving |
| Biotin | 0.002 | g/l | — |
| Glucose*H2O | 20 | g/l | — |
| Kanamycin | 50 | μg/mL | |

CGXII Minimal medium for fermentations in bioreactor

| Component | Concentration | | Comment |
|---|---|---|---|
| KH₂PO₄ | 2 | g/l | — |
| K₂HPO₄ | 2 | g/l | — |
| (NH₄)₂SO₄ | 5 | g/l | — |
| CaCl₂*2H₂O | 0.02 | g/l | — |
| MgSO₄*7H₂O | 1 | g/l | — |

—

Adjust pH to 7.0 with KOH and autoclave

| | | | |
|---|---|---|---|
| Trace element stock solution (1000x) | 1 | mL/L | Addition after autoclaving |
| Biotin | 0.002 | g/l | — |
| Polypropylene glycol | 0.005% | v/v | — |
| Glucose*H2O | 20 | g/l | — |
| Kanamycin | 50 | μg/mL | |

Composition of the trace element stock solution used (1000x)

| Component | Concentration | |
|---|---|---|
| CuSO₄*5H₂O | 0.2 | g/l |
| ZnSO₄*7H₂O | 1 | g/l |
| NiCl₂*6H₂O | 0.02 | g/l |
| MnSO₄*H₂O | 10 | g/l |
| FeSO₄*7H₂O | 10 | g/l |

### Results

Shaking flask cultures were carried out with the production strain C. glutamicum Δppc ΔtrpD::trpD5, which had been transformed with different expression plasmids. Table 3 shows product titers each measured over a 48 hour culture period. It can be seen that both the empty plasmid control and expression plasmids bearing aroF^{wt} or aro^{E154N} exhibited no effect on product formation in the strain background. Only the production strain with aroG^{new} expression plasmid showed a significant increase in the final titer by 54-72% (see Table 3).

TABLE 3
Product concentrations (g/L) from shaking flask cultures with different expression
plasmids. The measured values are mean values from two biological replicates. Measuring
points with a standard deviation greater than 5% were marked with an *.

| | Culture time (h) | | | | | |
|---|---|---|---|---|---|---|
| Production strain | 5 | 7 | 9 | 12 | 24 | 48 |
| C. glutamicum Δppc ΔtrpD::trpD5 | 0.018 | 0.056 | 0.111 | 0.215 | 0.612 | 0.551* |
| pEKEx2 | | | | | | |
| C. glutamicum Δppc ΔtrpD::trpD5 | 0.002 | 0.018 | 0.066* | 0.188* | 0.616 | 0.615 |
| pEKEx2_aroF^{wt} | | | | | | |
| C. glutamicum Δppc ΔtrpD::trpD5 | 0.002* | 0.024* | 0.067 | 0.159* | 0.586 | 0.557* |
| pEKEx2_aroF^{E154N} | | | | | | |
| C. glutamicum Δppc ΔtrpD::trpD5 | 0.043 | 0.087 | 0.138 | 0.244 | 0.871 | 0.947 |
| pEKEx2_aroG^{new} | | | | | | |

In order to characterize the positive effect of the gene variant aroG^{new} more precisely, fermentations were carried out in which the production strain C. glutamicum Δppc ΔtrpD::trpD5 ΔaroF was transformed in each case with the expression plasmids shown in Table 3. As shown in Table 4, the transformation with the expression plasmid bearing aroG^{new} has a positive effect on cell growth, substrate uptake, product titers and product yields (see Table 4). Based on the final product yield, the aroG^{new} expression achieved an improvement of 44-48%.

TABLE 4
Fermentative characterization of a production strain
with different expression plasmids in a bioreactor.

| | | | | |
|---|---|---|---|---|
| Time | Dry | Glucose | oAB | Product yield |
| (h) | weight (g) | consumption (g) | (g/L) | (g oAB/g glucose) |

C. glutamicum Δppc ΔtrpD::trpD5 ΔaroF pEKEx2

| | | | | |
|---|---|---|---|---|
| 0.0 | −0.02 | 0.00 | 0.00 | 0.000 |
| 5.6 | 0.02 | 0.20 | 0.05 | 0.022 |
| 20.6 | 0.11 | 0.68 | 0.53 | 0.071 |
| 24.2 | 0.19 | 0.93 | 0.62 | 0.061 |
| 28.9 | 0.38 | 1.46 | 1.19 | 0.073 |
| 46.1 | 1.54 | 4.99 | 4.32 | 0.084 |
| 49.9 | 1.75 | 5.18 | 1.99 | 0.044 |
| 53.8 | 2.09 | 6.72 | 4.40 | 0.067 |
| 68.8 | 4.38 | 12.70 | 4.38 | 0.041 |
| 70.6 | 4.76 | 13.67 | 4.57 | 0.041 |

C. glutamicum Δppc ΔtrpD::trpD5 ΔaroF pEKEx2-aroF^{wt}

| | | | | |
|---|---|---|---|---|
| 0.0 | −0.02 | 0.00 | 0.01 | 0.000 |
| 5.6 | 0.01 | 0.15 | 0.06 | 0.040 |
| 20.6 | 0.08 | 0.62 | 0.57 | 0.085 |
| 24.2 | 0.16 | 0.88 | 0.66 | 0.069 |
| 28.9 | 0.35 | 1.32 | 1.47 | 0.100 |
| 46.1 | 1.40 | 4.56 | 4.89 | 0.103 |
| 49.9 | 1.62 | 5.37 | 2.85 | 0.054 |
| 53.8 | 1.89 | 6.76 | 4.84 | 0.072 |
| 68.8 | 3.86 | 12.50 | 4.44 | 0.042 |
| 70.6 | 4.52 | 13.87 | 4.68 | 0.040 |

C. glutamicum Δppc ΔtrpD::trpD5 ΔaroF pEKEx2-aroF^{E154N}

| | | | | |
|---|---|---|---|---|
| 0.0 | −0.05 | 0.00 | 0.00 | 0.000 |
| 5.6 | 0.00 | 0.17 | 0.04 | 0.023 |
| 20.6 | 0.12 | 0.68 | 0.49 | 0.066 |
| 24.2 | 0.23 | 0.94 | 0.71 | 0.068 |
| 28.9 | 0.47 | 1.50 | 1.47 | 0.089 |
| 46.1 | 1.48 | 4.76 | 4.23 | 0.085 |
| 49.9 | 1.64 | 5.61 | 3.87 | 0.068 |
| 53.8 | 1.98 | 6.46 | 4.86 | 0.076 |
| 68.8 | 3.94 | 12.54 | 4.45 | 0.041 |
| 70.6 | 4.62 | 13.42 | 4.60 | 0.040 |

C. glutamicum Δppc ΔtrpD::trpD5 ΔaroF pEKEx2-aroG^{new}

| | | | | |
|---|---|---|---|---|
| 0.0 | −0.03 | 0.00 | 0.00 | 0.000 |
| 5.6 | 0.04 | 0.17 | 0.07 | 0.036 |
| 20.6 | 0.36 | 1.05 | 1.26 | 0.111 |
| 24.2 | 0.56 | 1.59 | 1.90 | 0.110 |
| 28.9 | 0.94 | 2.34 | 3.88 | 0.157 |
| 46.1 | 2.22 | 6.63 | 6.13 | 0.094 |
| 49.9 | 2.62 | 7.88 | 6.06 | 0.081 |
| 53.8 | 3.05 | 9.35 | 8.46 | 0.099 |
| 68.8 | 6.45 | 18.03 | 8.46 | 0.061 |
| 70.6 | 7.05 | 20.17 | 8.96 | 0.059 |

Since the results shown above demonstrate that the expression of aroF has no effect on the measured parameters, the strain C. glutamicum Δppc ΔtrpD::trpD5 was subsequently used for the investigation of further AroG variants. In this case, the strain C. glutamicum Δppc ΔtrpD::trpD5 was transformed with pEKEx2_aroG^{D146N} or pEKEx2_aroG^{S180F} for the expression of the gene variants aroG^{D146N} or aroG^{S180F} respectively from E. coli. Table 5 clearly shows that growth, substrate consumption and product titers are noticeably lower than the measured values of the strain with aroG^{new} expression plasmid (see Tables 4 and 5). It should be emphasized that up to a fermentation time of 28.9 h, the yield with plasmid-based aroG^{new} expression is also noticeably higher than the yield of all other DAHPS gene variants tested (see Tables 4 and 5). At this time point, the product yield with aroG^{new} expression 635% and 343% higher than the expression of aroG^{D146N} and aroGs^{S180F} respectively.

TABLE 5
Fermentative characterization of a production strain with
different expression plasmids in a bioreactor. The values
shown are mean values from two biological replicates.

| | | | | |
|---|---|---|---|---|
| Time | Dry | Glucose | oAB | Product yield |
| (h) | weight (g) | consumption (g) | (g/L) | (g oAB/g glucose) |

C. glutamicum Δppc ΔtrpD::trpD5 pEKEx2-aroG^{D146N}

| | | | | |
|---|---|---|---|---|
| 0.0 | 0.02 | 0.00 | 0.00 | — |
| 5.6 | 0.07 | 1.31 | 0.02 | 0.001 |
| 22.0 | 0.27 | 1.82 | 0.34 | 0.018 |
| 24.2 | 0.28 | 1.90 | 0.39 | 0.020 |
| 29.0 | 0.32 | 2.09 | 0.54 | 0.025 |
| 46.7 | 0.66 | 2.69 | 1.47 | 0.051 |
| 50.6 | 0.75 | 2.94 | 1.73 | 0.055 |
| 53.3 | 0.83 | 2.99 | 1.93 | 0.060 |
| 69.6 | 1.14 | 4.10 | 3.11 | 0.070 |
| 70.5 | 1.16 | 4.20 | 3.18 | 0.070 |

C. glutamicum Δppc ΔtrpD::trpD5 pEKEx2-aroG^{S180F}

| | | | | |
|---|---|---|---|---|
| 0.0 | 0.01 | 0.00 | 0.00 | — |
| 5.6 | 0.11 | 0.84 | 0.03 | 0.003 |
| 22.0 | 0.30 | 1.58 | 0.51 | 0.032 |
| 24.2 | 0.35 | 1.71 | 0.63 | 0.036 |
| 29.0 | 0.50 | 2.03 | 0.95 | 0.046 |
| 46.7 | 1.26 | 3.78 | 2.89 | 0.077 |
| 50.6 | 1.38 | 4.33 | 3.45 | 0.081 |
| 53.3 | 1.52 | 4.80 | 3.83 | 0.081 |
| 69.6 | 2.97 | 9.08 | 6.59 | 0.077 |
| 70.5 | 3.10 | 9.57 | 6.79 | 0.076 |

### DISCUSSION

The experiments described show that the introduction of the aroG^{new} gene variant has a surprising advantage for the production strains in terms of growth, substrate uptake, product formation and product yield. These observations were solely due to the modifications of the E. coli-based aroG gene variant aroG^{nww} in the C. glutamicum strain background and could not be replicated with other variants of DAHP synthetases investigated.

Sequence Listing Information:

DTD Version: V1_3
File Name: 2021P30312_Sequence_Listing_PCT.xml
Software Name: WIPO Sequence
Software Version: 2.2.0
Production Date: 2022-11-29
General Information:
Current application/Applicant file reference: 2021P30312EP
Earliest priority application/IP Office: EP
Earliest priority application/Application number: 21215753.1
Earliest priority application/Filing date: 2021-12-17
Applicant name: Covestro Deutschland AG
Applicant name/Language: de
Invention title: Particularly suitable
3-desoxyarabinoheptulosanate-7-phosphate synthase for the fermentative
reparation of ortho-aminobenzoic acid (en)
Sequence Total Quantity: 20
Sequences:
Sequence Number (ID): 1
Length: 1070
Molecule Type: DNA
Features Location/Qualifiers:
-misc_feature, 1..1070
>note, Expression construct
-source, 1..1070
>mol_type, other DNA
>organism, synthetic construct
Residues:

| | |
|---|---|
| gccctgcagg taaaaaaagg attgtgactt ctccagcaac actgaaagtt ctcaacgcct | 60 |
| acttggataa ccccactcca accctggagg aggcaattga ggtgttcacc ccgctgaccg | 120 |
| tgggtgaata cgatgacgtg cacatcgcag cgctgcttgc caccatccgt actcgcggtg | 180 |
| agcagttcgc tgatattgcc ggcgctgcca aggcgttcct cgcggcggct cgtccgttcc | 240 |
| cgattactgg cgcaggtttg ctagattccg ctggtactgg tggcgacggt gccaacacca | 300 |
| tcaacatcac caccggcgca tccctgatcg cagcatccgg tggagtgaag ctggttaagc | 360 |
| acggcaaccg ttcggtgagc tccaagtccg gctccgccga tgtgctggaa gcgctgaata | 420 |
| ttcctttggg ccttgatgtg gatcgtgctg tgaagtggtt cgaagcgtcc aacttcacct | 480 |
| tcctgttcgc acctgcgtac aaccctgcga ttgcgcatgt gcagccggtt cgccaggcgc | 540 |
| tgaaattccc caccatcttc aacacgcttg gaccattgct gtccccggcg cgcccggagc | 600 |
| gtcagatcat gggcgtggcc aatgccaatc atggacagct catcgccgag gtcttccgcg | 660 |
| agttgggccg tacacgcgcg cttgttgtgc atggcgcagg caccgatgag atcgcagtcc | 720 |
| acggcaccac cttggtgtgg gagcttaaag aagacggcac catcgagcat tacaccatcg | 780 |
| agcctgagga ccttggcctt ggccgctaca cccttgagga tctcgtaggt ggcctcggca | 840 |
| ctgagaacgc cgaagctatg cgcgctactt tcgcgggcac cggccctgat gcacaccgtg | 900 |
| atgcgttggc tgcgtccgca ggtgcgatgt tctacctcaa cggcgatgtc gactccttga | 960 |
| aagatggtgc acaaaaggcg ctttccttgc ttgccgacgg caccacccag gcatggttgg | 1020 |
| ccaagcacga agagatcgat tactcagaaa aggagtcttc caatgactag | 1070 |

Sequence Number (ID): 2
Length: 1070
Molecule Type: DNA
Features Location/Qualifiers:
-misc_feature, 1..1070
>note, Expression construct
-source, 1..1070
>mol_type, other DNA
>organism, synthetic construct
Residues:

| | |
|---|---|
| gccctgcagg taaaaaaagg attttgactt ctccagcaac actgaaagtt ctcaacgcct | 60 |
| acttggataa ccccactcca accctggagg aggcaattga ggtgttcacc ccgctgaccg | 120 |
| tgggtgaata cgatgacgtg cacatcgcag cgctgcttgc caccatccgt actcgcggtg | 180 |
| agcagttcgc tgatattgcc ggcgctgcca aggcgttcct cgcggcggct cgtccgttcc | 240 |
| cgattactgg cgcaggtttg ctagattccg ctggtactgg tggcgacggt gccaacacca | 300 |
| tcaacatcac caccggcgca tccctgatcg cagcatccgg tggagtgaag ctggttaagc | 360 |
| acggcaaccg ttcggtgagc tccaagtccg gctccgccga tgtgctggaa gcgctgaata | 420 |
| ttcctttggg ccttgatgtg gatcgtgctg tgaagtggtt cgaagcgtcc aacttcacct | 480 |
| tcctgttcgc acctgcgtac aaccctgcga ttgcgcatgt gcagccggtt cgccaggcgc | 540 |
| tgaaattccc caccatcttc aacacgcttg gaccattgct gtccccggcg cgcccggagc | 600 |
| gtcagatcat gggcgtggcc aatgccaatc atggacagct catcgccgag gtcttccgcg | 660 |
| agttgggccg tacacgcgcg cttgttgtgc atggcgcagg caccgatgag atcgcagtcc | 720 |
| acggcaccac cttggtgtgg gagcttaaag aagacggcac catcgagcat tacaccatcg | 780 |
| agcctgagga ccttggcctt ggccgctaca cccttgagga tctcgtaggt ggcctcggca | 840 |
| ctgagaacgc cgaagctatg cgcgctactt tcgcgggcac cggccctgat gcacaccgtg | 900 |
| atgcgttggc tgcgtccgca ggtgcgatgt tctacctcaa cggcgatgtc gactccttga | 960 |
| aagatggtgc acaaaaggcg ctttccttgc ttgccgacgg caccacccag gcatggttgg | 1020 |
| ccaagcacga agagatcgat tactcagaaa aggagtcttc caatgactag | 1070 |

Sequence Number (ID): 3
Length: 348
Molecule Type: AA
Features Location/Qualifiers:
-source, 1..348
>mol_type, protein
>organism, Corynebacterium glutamicum
Residues:

| | |
|---|---|
| MTSPATLKVL NAYLDNPTPT LEEAIEVFTP LTVGEYDDVH IAALLATIRT RGEQFADIAG | 60 |
| AAKAFLAAAR PFPITGAGLL DSAGTGGDGA NTINITTGAS LIAASGGVKL VKHGNRSVSS | 120 |
| KSGSADVLEA LNIPLGLDVD RAVKWFEASN FTFLFAPAYN PAIAHVQPVR QALKFPTIFN | 180 |
| TLGPLLSPAR PERQIMGVAN ANHGQLIAEV FRELGRTRAL VVHGAGTDEI AVHGTTLVWE | 240 |
| LKEDGTIEHY TIEPEDLGLG RYTLEDLVGG LGTENAEAMR ATFAGTGPDA HRDALAASAG | 300 |
| AMFYLNGDVD SLKDGAQKAL SLLADGTTQA WLAKHEEIDY SEKESSND | 348 |

Sequence Number (ID): 4
Length: 919
Molecule Type: AA
Features Location/Qualifiers:
-source, 1..919
>mol_type, protein
>organism, Corynebacterium glutamicum
Residues:

| | |
|---|---|
| MTDFLRDDIR FLGQILGEVI AEQEGQEVYE LVEQARLTSF DIAKGNAEMD SLVQVFDGIT | 60 |
| PAKATPIARA FSHFALLANL AEDLYDEELR EQALDAGDTP PDSTLDATWL KLNEGNVGAE | 120 |
| AVADVLRNAE VAPVLTAHPT ETRRRTVFDA QKWITTHMRE RHALQSAEPT ARTQSKLDEI | 180 |
| EKNIRRRITI LWQTALIRVA RPRIEDEIEV GLRYYKLSLL EEIPRINRDV AVELRERFGE | 240 |
| GVPLKPVVKP GSWIGGDHDG NPYVTAETVE YSTHRAAETV LKYYARQLHS LEHELSLSDR | 300 |
| MNKVTPQLLA LADAGHNDVP SRVDEPYRRA VHGVRGRILA TTAELIGEDA VEGVWFKVFT | 360 |
| PYASPEEFLN DALTIDHSLR ESKDVLIADD RLSVLISAIE SFGFNLYALD LRQNSESYED | 420 |
| VLTELFERAQ VTANYRELSE AEKLEVLLKE LRSPRPLIPH GSDEYSEVTD RELGIFRTAS | 480 |
| EAVKKFGPRM VPHCIISMAS SVTDVLEPMV LLKEFGLIAA NGDNPRGTVD VIPLFETIED | 540 |
| LQAGAGILDE LWKIDLYRNY LLQRDNVQEV MLGYSDSNKD GGYFSANWAL YDAELQLVEL | 600 |
| CRSAGVKLRL FHGRGGTVGR GGGPSYDAIL AQPRGAVQGS VRITEQGEII SAKYGNPETA | 660 |
| RRNLEALVSA TLEASLLDVS ELTDHQRAYD IMSEISELSL KKYASLVHED QGFIDYETQS | 720 |
| TPLQEIGSLN IGSRPSSRKQ TSSVEDLRAI PWVLSWSQSR VMLPGWFGVG TALEQWIGEG | 780 |
| EQATQRIAEL QTLNESWPFF TSVLDNMAQV MSKAELRLAK LYADLIPDTE VAERVYSVIR | 840 |
| EEYFLTKKMF CVITGSDDLL DDNPLLARSV QRRYPYLLPL NVIQVEMMRR YRKGDQSEQV | 900 |
| SRNIQLTMNG LSTALRNSG | 919 |

Sequence Number (ID): 5
Length: 2760
Molecule Type: DNA
Features Location/Qualifiers:
-source, 1..2760
>mol_type, genomic DNA
>organism, Corynebacterium glutamicum
Residues:

| | |
|---|---|
| atgactgatt ttttacgcga tgacatcagg ttcctcggtc aaatcctcgg tgaggtaatt | 60 |
| gcggaacaag aaggccagga ggtttatgaa ctggtcgaac aagcgcgcct gacttctttt | 120 |
| gatatcgcca agggcaacgc cgaaatggat agcctggttc aggttttcga cggcattact | 180 |
| ccagccaagg caacaccgat tgctcgcgca ttttcccact tcgctctgct ggctaacctg | 240 |
| gcggaagacc tctacgatga agagcttcgt gaacaggctc tcgatgcagg cgacacccct | 300 |
| ccggacagca ctcttgatgc cacctggctg aaactcaatg agggcaatgt tggcgcagaa | 360 |
| gctgtggccg atgtgctgcg caatgctgag gtggcgccgg ttctgactgc gcacccaact | 420 |
| gagactcgcc gccgcactgt ttttgatgcg caaaagtgga tcaccaccca catgcgtgaa | 480 |
| cgccacgctt tgcagtctgc ggagcctacc gctcgtacgc aaagcaagtt ggatgagatc | 540 |
| gagaagaaca tccgccgtcg catcaccatt ttgtggcaga ccgcgttgat tcgtgtggcc | 600 |
| cgcccacgta tcgaggacga gatcgaagta gggctgcgct actacaagct gagccttttg | 660 |
| gaagagattc cacgtatcaa ccgtgatgtg gctgttgagc ttcgtgagcg tttcggcgag | 720 |
| ggtgttcctt tgaagcccgt ggtcaagcca ggttcctgga ttggtggaga ccacgacggt | 780 |
| aacccttatg tcaccgcgga aacagttgag tattccactc accgcgctgc ggaaaccgtg | 840 |
| ctcaagtact atgcacgcca gctgcattcc ctcgagcatg agctcagcct gtcggaccgc | 900 |
| atgaataagg tcaccccgca gctgcttgcg ctggcagatg cagggcacaa cgacgtgcca | 960 |
| agccgcgtgg atgagcctta tcgacgcgcc gtccatggcg ttcgcggacg tatcctcgcg | 1020 |
| acgacggccg agctgatcgg cgaggacgcc gttgagggcg tgtggttcaa ggtctttact | 1080 |
| ccatacgcat ctccggaaga attcttaaac gatgcgttga ccattgatca ttctctgcgt | 1140 |
| gaatccaagg acgttctcat tgccgatgat cgtttgtctg tgctgatttc tgccatcgag | 1200 |
| agctttggat tcaaccttta cgcactggat ctgcgccaaa actccgaaag ctacgaggac | 1260 |
| gtcctcaccg agcttttcga acgcgcccaa gtcaccgcaa actaccgcga gctgtctgaa | 1320 |
| gcagagaagc ttgaggtgct gctgaaggaa ctgcgcagcc ctcgtccgct gatcccgcac | 1380 |
| ggttcagatg aatacagcga ggtcaccgac cgcgagctcg gcatcttccg caccgcgtcg | 1440 |
| gaggctgtta agaaattcgg gccacggatg gtgcctcact gcatcatctc catggcatca | 1500 |
| tcggtcaccg atgtgctcga gccgatggtg ttgctcaagg aattcggact catcgcagcc | 1560 |
| aacggcgaca acccacgcgg caccgtcgat gtcatcccac tgttcgaaac catcgaagat | 1620 |
| ctccaggccg gcgccggaat cctcgacgaa ctgtggaaaa ttgatctcta ccgcaactac | 1680 |
| ctcctgcagc gcgacaacgt ccaggaagtc atgctcggtt actccgattc caacaaggat | 1740 |
| ggcggatatt tctccgcaaa ctgggcgctt tacgacgcgg aactgcagct cgtcgaacta | 1800 |
| tgccgatcag ccggggtcaa gcttcgcctg ttccacggcc gtggtggcac cgtcggccgc | 1860 |
| ggtggcggac cttcctacga cgcgattctt gcccagccca ggggggctgt ccaaggttcc | 1920 |
| gtgcgcatca ccgagcaggg cgagatcatc tccgctaagt acggcaaccc cgaaaccgcg | 1980 |
| cgccgaaacc tcgaagccct ggtctcagcc acgcttgagg catcgcttct cgacgtctcc | 2040 |
| gaactcaccg atcaccaacg cgcgtacgac atcatgagtg agatctctga gctcagcttg | 2100 |
| aagaagtacg cctccttggt gcacgaggat caaggcttca tcgattactt cacccagtcc | 2160 |
| acqccgctgc aggagattgg atccctcaac atcggatcca ggccttcctc acgcaagcag | 2220 |
| acctcctcgg tggaagattt gcgagccatc ccatgggtgc tcagctggtc acagtctcgt | 2280 |
| gtcatgctgc caggctggtt tggtgtcgga accgcattag agcagtggat tggcgaaggg | 2340 |
| gagcaggcca cccaacgcat tgccgagctg caaacactca atgagtcctg gccatttttc | 2400 |
| acctcagtgt tggataacat ggctcaggtg atgtccaagg cagagctgcg tttggcaaag | 2460 |
| ctctacgcag acctgatccc agatacggaa gtagccgagc gagtctattc cgtcatccgc | 2520 |
| gaggagtact tcctgaccaa gaagatgttc tgcgtaatca ccggctctga tgatctgctt | 2580 |
| gatgacaacc cacttctcgc acgctctgtc cagcgccgat acccctacct gcttccactc | 2640 |
| aacgtgatcc aggtagagat gatgcgacgc taccgaaaag gcgaccaaag cgagcaagtg | 2700 |
| tcccgcaaca ttcagctgac catgaacggt ctttccactg cgctgcgcaa ctccggctag | 2760 |

Sequence Number (ID): 6
Length: 185
Molecule Type; AA
Features Location/Qualifiers;
-source, 1..185
>mol_type, protein
>organism, Corynebacterium glutamicum
Residues:

| | |
|---|---|
| MNDQIHLDHQ SDDTSECSCP IVVLVGLPGA GKSTIGRRLA RALNTELVDS DELIERATGK | 60 |
| ACGAVFSELG EPAFRELEAI HVAEALKSSG VVSLGGGSVL TESTRELLKG QDVVWIDVPV | 120 |
| EEGIRRTANE RSRPVLQAAD PAEHYRNLVK VRTPLYEEVA TYRLRINNRS PQQVVAAVLH | 180 |
| HLEID | 185 |

Sequence Number (ID): 7 Length: 174
Molecule Type: AA
Features Location/Qualifiers:
-source, 1..174
>mol_type, protein
>organism, Escherichia coli
Residues:

| | |
|---|---|
| MTQPLFLIGP RGCGKTTVGM ALADSLNRRF VDTDQWLQSQ LNMTVAEIVE REEWAGFRAR | 60 |
| ETAALEAVTA PSTVIATGGG IILTEFNRHF MQNNGIVVYL CAPVSVLVNR LQAAPEEDLR | 120 |
| PTLTGKPLSE EVQEVLEERD ALYREVAHII IDATNEPSQV ISEIRSALAQ TINC | 174 |

Sequence Number (ID): 8
Length: 179
Molecule Type: DNA
Features Location/Qualifiers:
-misc_feature, 1..179
>note, Promoter sequence
-source, 1..179
>mol_type, other DNA
>organism, synthetic construct
Residues:

| | |
|---|---|
| cacagggtag ctggtagttt gaaaatcaac gccgttgccc ttaggattca gtaactggca | 60 |
| cattttgtaa tgcgctagat ctgtgtgctc agtcttccag gctgcttatc acagtgaaag | 120 |
| caaaaccaat tcgtggetgc gaaagtcgta gccaccacga agtccaggag gacatacat | 179 |

Sequence Number (ID): 9
Length: 430
Molecule Type: AA
Features Location/Qualifiers:
-source, 1..430
>mol_type, protein
>organism, Escherichia coli
Residues:

| | |
|---|---|
| MVFVSDSSIS LPIWDAPRAR GPIVSDLAIP GSKSITNRAL ILAALASTPS TIIDVLRSRD | 60 |
| TDLMTDGLRS LGITITEEAV DRYRVEPGQL SAGSVECGLA GTVMRFLPPV AAFADGPVHF | 120 |
| DGDPQARVRP MTSILDALRS LGVEVDNNNL PFTVNAGEVP EGGVVEIDAS GSSQFVSGLL | 180 |
| LSAPREKNGV TVKHVGGRLP SMPHIEMTVD MLRSAGIEIE ESENQWVVHP GEILGRTWRI | 240 |
| EPDLSNATPF LAAAAVTGGT IKINHWPIKT TOPGDAIRSI LERMGCEVEL VAQGEGYDLS | 300 |
| VTGPVALKGI EIDMSDIGEL TPTVAALAAL ASTESRLTGI AHLRGHETDR LAALTAEINK | 360 |
| LGGKCTELKD GLLIEPASLH GGVWHSYADH RMATAGAIIG LAVDGVQVED IKTTSKTFPG | 420 |
| FENVWEEMVG | 430 |

Sequence Number (ID): 10
Length: 410
Molecule Type: AA
Features Location/Qualifiers:
-source, 1..410
>mol_type, protein
>organism, Corynebacterium glutamicum
Residues:

| | |
|---|---|
| MLGMLRWTTA GESHGOALIA TVEHMPAGVP VTKDEVSYQL ARRRLGYGRG ARMKFEQDAL | 60 |
| TELTGIRHGL TLGSPISIMI GNTEWDKWTT IMSSDALDME DPDNVAAMSS GRGAKLTRPR | 120 |
| PGHADYAGML KYGEDDARNV LERSSARETA ARVAAATVAR SFLRETLGVE VLSHVISIGA | 180 |
| SEPYTGAEPT FADIQAIDDS PVRAFGKDAE ESMIAEIEAA KKAGDTLGGI VEVIVEGLPI | 240 |
| GLGSHISGED RLDAQIAAAL MGIQAIKGVE IGDGFEEARR RGSEAHDEVF LDDNGVYRNT | 300 |
| NRAGGLEGGM TNGETLRVRA GMKPISTVPR ALKTIDMENG KAATGIHORS DVCAVPAAGV | 360 |
| VAEAMVTLVL ARAVLQKFGG DSLSETKSNI DTYLKNIEER MKFEGLEDGA | 410 |

Sequence Number (ID): 11
Length: 350
Molecule Type: AA
Features Location/Qualifiers;
-REGION, 1..350
>note, modified enzyme
-source, 1..350
>mol_type, protein
>organism, synthetic construct
Residues:

| | |
|---|---|
| MNYQNDDLRI KEIKELLPPV ALLEKFPATE NAANTVAHAR KAIHKILKGN DDRLLVVIGP | 60 |
| CSIHDPVAAK EYATRVLALR EELKDELEIV MRVYFEKPRT TVGWKGLIND PHMDNSFQIN | 120 |
| DGLRIARKLL LDINDSGLPA AGEFLDMITP QYLADLMSWG AIGARTTESQ VHRELASGLS | 180 |
| CPVGFKNGTD GTIKVAIDAI NAAGAPHCFL FVTKWGHSAI VNTSGNGDCH IILRGGKEPN | 240 |
| YSAKHVAEVK EGLNKAGLPA QVMIDESHAN SSKQFKKQMD VCADVCQQIA GGEKAIIGVM | 300 |
| VESHLVEGNQ SLESGEPLAY GKSITDACIG WEDTDALLRQ LANAVKARRG | 350 |

Sequence Number (ID): 12
Length: 446
Molecule Type: AA
Features Location/Qualifiers:
-source, 1..446
>mol_type, protein
>organism, Xanthomonas campestris
Residues:

| | |
|---|---|
| MSNTVFIGAK EYFPGIGKIG FEGRDSDNPL AFKVYDANKQ VAGKTMAEHL RFAVAYWHSF | 60 |
| CGNGADPFGP GTRAYPWDVG NTALARAEAK SDAAFEFFTK LGVPYYCFHD IDLAPDADDI | 120 |
| GEYENNLKHM VGIAKQRQAD TGVKLLWGTA NLFSHPRYMN GASTNPDFNV VARAAVQVKA | 180 |
| AIDATVELGG ENYVFWGGRE GYACLHNTQM KREQDNMARE LTLARDYGRA IGFTGNFLIE | 240 |
| PKPMEPMKHQ YDFDSATVIG FLRQHGLDQD FKLNIEANHA TLSGHSFEHD LQVASDAGLL | 300 |
| GSIDANRGNP QNGWDTDQFP TDLYDTVGAM LVVLRQGGLA PGGLNFDAKV RRESSDPQDL | 350 |
| FLAHIGGMDA FARGLEVANA LLTSSPLETW RAQRYASFDS GAGADFANGT STLADLAKYA | |
| AGKGEPTQVS GRQEAYENLI NQYLTR | |

Sequence Number (ID): 13
Length: 463
Molecule Type: AA
Features Location/Qualifiers:
-REGION, 1..463
>note, modified enzyme
-source, 1..463
>mol_type, protein
>organism, synthetic construct
Residues:

| | |
|---|---|
| MSNTVFIGAK EYFPGIGKIG FEGRDSDNPL AFKVYDANKQ VAGKTMAEHL RFAVAYWHSF | 60 |
| CGNGADPFGP GTRAYPWDVG NTALARAEAK SDAAFEFFTK LGVPYYCFHD IDLAPDADDI | 120 |
| GEYENNLKHM VGIAKQRQAD TGVKLLWGTA NLFSHPRYMN GASTNPDENV VARAAVQVKA | 180 |
| AIDATVELGG ENYVFWGGRE GYACLHNTQM KREQDNMARE LTLARDYGRA IGFTGNFLIE | 240 |
| PKPMEPMKHQ YDFDSATVIG FLRQHGLDQD FKLNIEANHA TLSGHSFEHD LQVASDAGLL | 300 |
| GSIDANRGNP QNGWDTDQFP TDLYDTVGAM LVVLRQGGLA PGGLNFDAKV RRESSDPQDL | 360 |
| FLAHIGGMDA FARGLEVANA LLTSSPLETW RAQRYASFDS GAGADFANGT STLADLAKYA | 420 |
| AGKGEPTQVS GROEAYENLI NOYLTRHRVA GSLKINAVAL RIQ | 463 |

Sequence Number (ID): 14
Length: 460
Molecule Type: AA
Features Location/Qualifiers:
source, 1..460
>mol_type, protein
>organism, Corynebacterium glutamicum
Residues:

| | |
|---|---|
| MALVLGIDSS TQSCKALLVD AATGQVIDEG RASHPSGSEV DPRAWIAALD QATEGLLERA | 60 |
| DAVSIAGQQH GMVALDENDE IVRPALLWND TRSAQAALDL NEEIGGDQAA VDATGSVYVA | 120 |
| SLTATKMRWM RDHEPENAAR TASVMLPHDE LTWHLMGRGR KVTDHGDASG TGYYSTRDRA | 180 |
| WRTDLAALAL GHEVELPELL APNAIAGTTP GGVKVAAGTG DNAAAALGLD LQPGDVSVSI | 240 |
| GTSGVAGMTV QHSVHDPSGL VTGFADATGA YFPLACTLNG APVLEFGRRI LGVEWEEFDA | 300 |
| LALAAQPGSG GVTLQPYLEG ERTPNRPAAR GVLAGLNCAT TREDFARATV EGLLLALDDA | 360 |
| VTALVEATGV PVQRIQLIGG GARSQAVREI APEIFGHEIV VPEPAEYVAL GAARQAAWAL | 420 |
| SGEATPPQWP TPGSDPHRAP KNTELSTRYA KLRAATQGWY | 460 |

Sequence Number (ID): 15
Length: 3302
Molecule Type: DNA
Features Location/Qualifiers:
-misc_feature, 1..3302
>note, Expression construct
-source, 1..3302
>mol_type, other DNA
>organism, synthetic construct
Residues:

| | |
|---|---|
| gatcccacag ggtagctggt agtttgaaaa tcaacgccgt tgcccttagg attcagtaac | 60 |
| tggcacattt tgtaatgcgc tagatctgtg tgctcagtct tccaggctgc ttatcacagt | 120 |
| gaaagcaaaa ccaattcgtg gctgcgaaag tcgtagccac cacgaagtcc aggaggacat | 180 |
| acagtatgac acaacctctt tttctgatcg ggcctcgggg ctgtggtaaa acaacggtcg | 240 |
| gaatggccct tgccgattcg cttaaccgtc ggtttgtcga taccgatcag tggttgcaat | 300 |
| cacagctcaa tatgacggtc gcggagatcg tcgaaaggga agagtgggcg ggatttcgcg | 360 |
| ccagagaaac ggcggcgctg gaagcggtaa ctgcgccatc caccgttatc gctacaggcg | 420 |
| gcggcattat tctgacggaa tttaatcgtc acttcatgca aaataacggg atcgtggttt | 480 |
| atttgtgtgc gccagtatca gtcctggtta accgactgca agctgcaccg gaagaagatt | 540 |
| tacggccaac cttaacggga aaaccgctga gcgaagaagt tcaggaagtg ctggaagaac | 600 |
| gcgatgcgct atatcgcgaa gttgcgcata ttatcatcga cgcaacaaac gaacccagcc | 660 |
| aggtgatttc tgaaattcgc agcgccctgg cacagacgat caattgttga caatctttag | 720 |
| aggagacaca acatggtctt tgtgtctgat tcgtctatct ctttgcccat ttgggatgct | 780 |
| ccgcgcgctc gcggccccat agtctcggac ctggctatcc ctggttccaa gtcgatcacc | 840 |
| aaccgcgccc tcatcttggc tgcgctcgca tcaactccat ccaccatcat tgatgtcctt | 900 |
| cgtagtcgtg ataccgatct catgactgat ggtctacgca gcctcggaat caccattact | 960 |
| gaagaggcag tcgatcgcta ccgcgttgag cccggacagt tgtctgctgg ctccgttgag | 1020 |
| tgtggtcttg ctggtacggt catgcgcttt ttgcctcctg ttgctgcttt cgctgatggt | 1080 |
| cctgttcatt ttgatggcga tcctcaagct cgtgttcgtc cgatgaccag cattttggat | 1140 |
| gcgctgcgtt cgcttggtgt ggaggtagac aacaacaatc tgcctttcac tgttaatgct | 1200 |
| ggtgaggtcc ctgagggtgg cgtggttgag attgatgctt ccggctcatc tcagtttgtt | 1260 |
| tctggtcttt tgctttcagc gcctcgtttt aaaaatggcg tcaccgttaa gcacgtcggt | 1320 |
| ggtcgtctgc cgagcatgcc qcatattgag atgaccgtcg atatgcttcg ttccgcaggc | 1380 |
| attgagatcg aagagtcaga aaatcagtgg gttgttcatc ctggtgagat cttgggtcgg | 1440 |
| acctggcgca ttgagccgga tctttctaat gcgactccgt tcctagctgc cgctgcggtc | 1500 |
| actggtggaa ccatcaagat taaccactgg ccaatcaaaa ctactcagcc tggcgatgct | 1560 |
| attcgttcga ttcttgagcg catgggctgc gaagttgagc tggttgctca gggtgaaggt | 1620 |
| tacgatctgt cggtgactgg tccggttgct ctcaagggca ttgagatcga tatgtccgat | 1680 |
| atcggtgagt tgacccctac cgtggcggcg ttggctgcgt tggcgtcgac agagtctcgt | 1740 |
| ttgaccggta ttgctcatct tcgtggccat gagacggatc gtttggctgc gttgactgcg | 1800 |
| gagatcaaca aacttggtgg aaagtgcact gagcttaagg atggtctgtt gattgagcct | 1860 |
| gcgtcgctgc acggtggtgt gtggcattca tatgctgatc accgtatggc tactgctggt | 1920 |
| gcgatcattg gcctcgcggt tgatggcgtt caggttgaag acattaagac cacttccaaa | 1980 |
| actttccctg gttttgaaaa tgtttgggag gagatggttg gctaggcgaa agtcgtagcc | 2040 |
| accacgaagt ccgaaaggag gcccttcaga tgctaggcat gcttcgatgg actacagcag | 2100 |
| gtgaatccca cggccaggcg cttatcgcca cggttgaaca catgccagca ggcgtgcccg | 2160 |
| tgactaaaga tgaggtctcg tatcaattgg cgcgccgacg ccttggatat ggtcgcggcg | 2220 |
| ctcgcatgaa gtttgagcaa gacgcgttga ccttcctcac cggcatccgc cacggcctca | 2280 |
| ctttgggtag ccccatctca atcatgatcg gcaacactga gtgggataag tggaccacca | 2340 |
| tcatgtcctc tgacgctttg gacatggaag acccagataa cgttgcggcg atgtcttcgg | 2400 |
| gtcggggcgc aaaactgact cgtccgcgtc caggccacgc tgattacgca ggcatgctca | 2460 |
| agtacggatt cgatgatgcc cgcaacgtgc tggagcgttc ttcagcccgt gagacggcag | 2520 |
| cacgcgtggc agcagcaacc gttgcgcgtt ccttcctgcg tgaaaccttg ggcgtggaag | 2580 |
| tgctttccca cgtaatttcc attggtgcgt ccgagcctta cactggcgcg gagccaacct | 2640 |
| ttgcagatat tcaagcaatc gatgattccc cagttcgtgc attcggtaaa gacgctgaag | 2700 |
| aatccatgat cgcggaaatc gaggccgcaa agaaagccgg cgataccctc ggtggcatcg | 2760 |
| tggaagtgat tgttgaaggc ctgcccatcg gtttgggctc acacatttct ggcgaagatc | 2820 |
| gcctcgatgc gcagatcgca gctgcactca tgggcattca ggccatcaag ggcgtggaaa | 2880 |
| tcggtgacgg tttcgaagaa gctcgtcgac gtggctccga agcccacgat gaagtgttcc | 2990 |
| tggatgacaa cggcgtatac cgcaacacca accgtgcagg tggcctcgaa ggcggcatga | 3000 |
| ccaacggtga aaccctgcgc gttcgtgctg gcatgaagcc aatttctact gtgcctcgcg | 3060 |
| ccctgaaaac cattgatatg gaaaacggca aggcagcaac cggaatccac cagcgttccg | 3120 |
| acgtgtgcgc tgttccagcc gccggtgtcg ttgcagaagc aatggtcacc ctggttctcg | 3180 |
| cccgcgcagt cctgcagaaa ttcggcggtg actccctgag cgaaaccaag agcaacattg | 3240 |
| acacctacct caaaaacatt gaggaacgaa tgaaattcga aggtttagag gatggagcgt | 3300 |
| aa | 3302 |

Sequence Number (ID): 16
Length: 3539
Molecule Type: DNA
Features Location/Qualifiers:
-misc_feature, 1..3539
>note, Expression construct
-source, 1. . 3539
>mol_type, other DNA
>organism, synthetic construct
Residues:

| | |
|---|---|
| cacagggtag ctggtagttt gaaaatcaac gccgttgccc ttaggattca gtaactggca | 60 |
| cattttgtaa tgcgctagat ctgtgtgctc agtcttccag gctgcttatc acagtgaaag | 120 |
| caaaaccaat tcgtggctgc gaaagtcgta gccaccacga agtccaggag gacatacatg | 180 |
| aaaggaggcc cttcagatgt ccaacaccgt gttcatcggc gcaaaggaat acttcccagg | 240 |
| catcggcaag atcggcttcg aaggccgcga ttccgataac ccactggcat tcaaggtgta | 300 |
| cgatgcaaac aagcaggtgg caggcaagac catggcagaa cacctgcgct tcgcagtggc | 360 |
| atactggcac tccttctgcg gcaacggcgc agatccattc ggcccaggca cccgcgcata | 420 |
| cccatgggat gtgggcaaca ccgcactggc acgcgcagaa gcaaagtccg atgcagcatt | 480 |
| cgaattcttc accaagctgg gcgtgccata ctactgcttc cacgatatcg atctggcacc | 540 |
| agatgcagat gatatcggcg aatacgaaaa caacctgaag cacatggtgg gcatcgcaaa | 600 |
| gcagcgccag gcagataccg gcgtgaagct gctgtggggc accgcaaacc tgttctccca | 660 |
| cccacgctac atgaacggcg catccaccaa cccagatttc aacgtggtgg cacgcgcagc | 720 |
| agtgcaggtg aaggcagcaa tcgatgcaac cgtggaactg ggcggcgaaa actacgtgtt | 780 |
| ctggggcggc cgcgaaggct acgcatgcct gcacaacacc cagatgaagc gcgaacagga | 840 |
| taacatggca cgcttcctga ccctggcacg cgattacggc cgcgcaatcg gcttcaccgg | 900 |
| caacttcctg atcgaaccaa agccaatgga accaatgaag caccagtacg atttcgattc | 960 |
| cgcaaccgtg atcggcttcc tgcgccagca cggcctggat caggatttca agctgaacat | 1020 |
| cgaagcaaac cacgcaaccc tgtccggcca ctccttcgaa cacgatctgc aggtggcatc | 1080 |
| cgatgcaggc ctgctgggct ccatcgatgc aaaccgcggc aacccacaga acggctggga | 1140 |
| taccgatcag ttcccaaccg atctgtacga taccgtgggc gcaatgctgg tggtgctgcg | 1200 |
| ccagggcggc ctggcaccag gcggcctgaa cttcgatgca aaggtgcgcc gcgaatcctc | 1260 |
| cgatccacag gatctgttcc tggcacacat cggcggcatg gatgcattcg cacgcggcct | 1320 |
| ggaagtggca aacgcactgc tgacctcctc cccactggaa acctggcgcg cacagcgcta | 1380 |
| cgcatccttc gattccggcg caggcgcaga tttcgcaaac ggcacctcca ccctggcaga | 1440 |
| tctggcaaag tacgcagcag gcaagggcga accaacccag gtgtccggcc gccaggaagc | 1500 |
| atacgaaaac ctgatcaacc agtacctgac ccgccacagg gtagctggta gtttgaaaat | 1560 |
| caacgccgtt gcccttagga ttcagtaact ggcacatttt gtaatgcgct agatctgtgt | 1620 |
| gctcagtctt ccaggctgct tatcacagtg aaagcaaaac caattcgtgg ctgcgaaagt | 1680 |
| cgtagccacc acgaagtcca ggaggacata catgaaagga ggcccttcag atggctttgg | 1740 |
| ttcttggaat cgatagttcc acccaatcct gcaaggcttt gcttgtcgat gccgccaccg | 1800 |
| gccaggttat cgacgaaggc cgcgcgagtc acccgagcgg gtcggaggta gatccacgtg | 1860 |
| cgtggatcgc tgcgctggat caagctaccg aggggttgtt agaacgcgcg gacgctgtat | 1920 |
| ctattgcagg ccagcagcac ggcatggtgg cgttggatga aaacgatgaa atcgttcgcc | 1980 |
| cggcgttgtt atggaatgac actcgttctg cccaggctgc gttggatctc aatgaggaga | 2040 |
| tcggcggcga tcaggctgcg gtagatgcca cgggaagtgt gtatgttgct tctttaactg | 2100 |
| ccaccaaaat gcggtggatg cgtgatcatg aaccagaaaa tgcagcgcgc acggcgtcgg | 2160 |
| tgatgttgcc tcatgatttc ctcacctggc atttgatggg acgcggacgc aaagtcaccg | 2220 |
| accatggtga tgcttctgga acgggctact acagcacgcg tgatcgtgcg tggcgcaccg | 2280 |
| atctagctgc cttggcgctg ggccatgagg tggaacttcc tgaactcctg gccccaaatg | 2340 |
| cgattgcggg aacaactcca ggtggagtga aagttgctgc aggcacggga gataatgctg | 2400 |
| cggctgcgct tggccttgat ttgcagcctg gtgatgtcag cgtgtcgatc ggcacctctg | 2460 |
| gcgttgccgg catgaccgtt caacatagcg tccacgatcc atctggtttg gtcactggtt | 2520 |
| tcgccgatgc cacgggtgcg tatttcccgc tggcctgcac gcttaatggc gcaccggtgt | 2580 |
| tggaattcgg ccgccgcatt ctgggcgtgg aatgggaaga gttcgatgcg cttgcactgg | 2640 |
| ctgctcaacc cggttcaggt ggcgtgacgc tccagcctta tttggagggc gagcgtacgc | 2700 |
| cgaatcgtcc cgcagcacgt ggcgttttgg ctggactaaa ctgtgcaacg acccgcgagg | 2760 |
| actttgcccg agcaactgtt gaaggcttgt tgttggcatt ggatgatgct gtaacggcgc | 2820 |
| tggttgaggc cacgggagtg cccgttcagc gcatccagct catcggtggc ggcgcgcgtt | 2880 |
| cacaggcggt tcgtgagatt gcccctgaga ttttcggcca tgagattgtg gttccagaac | 2940 |
| ccgctgaata tgtggcgttg ggtgcagctc gtcaggcggc atgggcgctg tcgggtgagg | 3000 |
| ccacgccacc gcagtggcca actcccggtt ccgatccgca ccgcgcacct aaaaacactg | 3060 |
| agctgagcac gcgttatgcg aagctgcgtg ctgcaacgca gggttggtac tagggctgtt | 3120 |
| ttggcggatg agagaagatt ttcagcctga tacagattaa atcagaacgc agaagcggtc | 3180 |
| tgataaaaca gaatttgcct ggcggcagta gcgcggtggt cccacctgac cccatgccga | 3240 |
| actcagaagt gaaacgccgt agcgccgatg gtagtgtggg gtctccccat gcgagagtag | 3300 |
| ggaactgcca ggcatcaaat aaaacgaaag gctcagtcga aagactgggc ctttcgtttt | 3360 |
| atctgttgtt tgtcggtgaa cgctctcctg agtaggacaa atccgccggg agcggatttg | 3420 |
| aacgttgcga agcaacggcc cggagggtgg cgggcaggac gcccgccata aactgccagg | 3480 |
| catcaaatta agcagaaggc catcctgacg gatggccttt ttgcgtttct acaaactct | 3539 |

Sequence Number (ID): 17
Length: 1101
Molecule Type: DNA
Features Location/Qualifiers:
-source, 1..1101
>mol_type, other DNA
>organism, Corynebacterium glutamicum
Residues:

| | |
|---|---|
| atgagttctc cagtctcact cgaaaacgcg gcgtcaacca gcaacaagcg cgtcgtggct | 60 |
| ttccacgagc tgcctagccc tacagatctc atcgccgcaa acccactgac accaaagcag | 120 |
| gcttccaagg tggagcagga tcgccaggac atcgctgata tcttcgctgg cgacgatgac | 180 |
| cgcctcgttg tcgttgtggg accttgctca gttcacgatc ctgaagcagc catcgattac | 240 |
| gcaaaccgcc tggctccgct ggcaaagcgc cttgatcagg acctcaagat tgtcatgcgc | 300 |
| gtgtacttcg agaagcctcg caccatcgtc ggatggaagg gattgatcaa tgatcctcac | 360 |
| ctcaacgaaa cctacgacat cccagagggc ttgcgcattg cgcgcaaagt gcttatcgac | 420 |
| gttgtgaacc ttgatctccc agtcggctgc gaattcctcg aaccaaacag ccctcagtac | 480 |
| tacgccgaca ctgtcgcatg gggagcaatc ggcgctcgta ccaccgaatc tcaggtgcac | 540 |
| cgccagctgg cttctgggat gtctatgcca attggtttca agaacggaac tgacggaaac | 600 |
| atccaggttg cagtcgacgc ggtacaggct gcccagaacc cacacttctt cttcggaacc | 660 |
| tccgacgacg gcgcgctgag cgtcgtggag accgcaggca acagcaactc ccacatcatt | 720 |
| ttgcgcggcg gtacctccgg cccgaatcat gatgcagctt cggtggaggc cgtcgtcgag | 780 |
| aagcttggtg aaaacgctcg tctcatgatc gatgcttccc atgctaactc cggcaaggat | 840 |
| catatccgac aggttgaggt tgttcgtgaa atcgcagagc agatttctgg cggttctgaa | 900 |
| gctgtggctg gaatcatgat tgagtccttc ctcgttggtg gcgcacagaa ccttgatcct | 960 |
| gcgaaattgc gcatcaatgg cggtgaaggc ctggtgtacg gacagtctgt gaccgataag | 1020 |
| tgcatcgata ttgacaccac catcgatttg ctcgctgagc tggccgcagc agtaagggaa | 1080 |
| cgccgagcag cagccaagta a | 1101 |

Sequence Number (ID): 18
Length: 366
Molecule Type; AA
Features Location/Qualifiers:
-REGION, 1..366
>note, Modified enzyme
-source, 1..366
>mol_type, protein
>organism, synthetic construct
Residues:

| | |
|---|---|
| MSSPVSLENA ASTSNKRVVA FHELPSPTDL IAANPLTPKQ ASKVEQDRQD IADIFAGDDD | 60 |
| RLVVVVGPCS VHDPEAAIDY ANRLAPLAKR LDQDLKIVMR VYFEKPRTIV GWKGLINDPH | 120 |
| LNETYDIPEG LRIARKVLID VVNLDLPVGC EFLNPNSPQY YADTVAWGAI GARTTESQVH | 180 |
| RQLASGMSMP IGFKNGTDGN IQVAVDAVQA AQNPHFFFGT SDDGALSVVE TAGNSNSHII | 240 |
| LRGGTSGPNH DAASVEAVVE KLGENARLMI DASHANSGKD HIRQVEVVRE IAEQISGGSE | 300 |
| AVAGIMIESF LVGGAQNLDP AKLRINGGEG LVYGQSVTDK CIDIDTTIDL LAELAAAVRE | 360 |
| RRAAAK | 366 |
| | 480 |

Sequence Number (ID): 19
Length: 1053
Molecule Type: DNA
Features Location/Qualifiers:
-source, 1..1053
>mol_type, other DNA
>organism, synthetic construct
Residues:

| | |
|---|---|
| atgaattatc agaacgacga cctgcgcatc aagaactgct aaagaaatta gcctccggtt | 60 |
| gcactgctgg aaaaatttcc ggcaaccgaa aatgcagcaa ataccgttgc acatgcacgt | 120 |
| aaagccattc acaaaattct gaaaggtaat gatgatcgcc tgctggttgt tattggtccg | 180 |
| tgtagcattc atgatccggt tgccgcaaaa gaatatgcaa cccgtctgct ggcactgcgt | 240 |
| gaagaactga aagatgaact ggaaattgtg atgcgcgtgt attttgaaaa accgcgtacc | 300 |
| accgttggtt ggaaaggtct gattaatgat ccgcacatgg ataacagctt tcagattaac | 360 |
| gatggtctgc gtattgcccg taaactgctg ctggatatta atgatagcgg tctgcctgca | 420 |
| gccggtgaat ttctgaatat gattacaccg cagtatctgg ccgatctgat gagctggggt | 480 |
| gcaattggtg cacgcaccac cgaaagccag gttcatcgtg aactggcaag cggtctgagc | 540 |
| tgtccggttg gctttaaaaa cggcaccgat ggcaccatta aagttgcaat tgatgcaatt | 600 |
| aatgcagcgg gtgcaccgca ttgttttctg agcgttacca aatggggtca tagcgcaatt | 660 |
| gttaatacca gcggtaatgg tgattgccat attattctgc gtggtggtaa agaaccgaat | 720 |
| tattcagcaa aacatgtggc cgaagttaaa gaaggtctga ataaagcagg tctgccagca | 480 |
| caggttatga ttgattttag ccatgcaaat agcagcaaac agttcaaaaa acaaatggat | 840 |
| gtttgtgccg atgtgtgtca gcagattgcc ggtggtgaaa aagcaattat tggtgttatg | 900 |
| gttgaaagcc atctggtgga aggtaatcag agcctggaaa gcggtgaacc gctggcctat | 960 |
| ggtaaaagca ttaccgatgc atgtattggt tgggaagata ccgatgccct gctgcgtcag | 1020 |
| ctggcaaatg cagtaaaagc gcgtcgcggg taa | 1053 |

Sequence Number (ID): 20
Length: 1053
Molecule Type: DNA
Features Location/Qualifiers;
-source, 1..1053
>mol_type, other DNA
>organism, synthetic construct
Residues:

| | |
|---|---|
| atgaactacc agaacgatga tctgcgcatc aaagagatca aagaactgct gccaccagtg | 60 |
| gcactgctgg aaaagttccc agcaaccgaa aacgcagcaa acaccgtggc acacgcacgc | 120 |
| aaggcaatcc acaagatcct gaagggcaac gatgaccgcc tgctcgtggt gatcggtcca | 180 |
| tgctccatcc acgatccagt ggcagcaaaa gaatacgcaa cccgcttgct ggcactgcgc | 240 |
| gaagaactga aggatgaact ggaaatcgtg atgcgcgtgt acttcgaaaa gccacgcacc | 300 |
| accgtcggct ggaagggcct gatcaacgat ccacacatgg ataattcctt ccagatcaac | 360 |
| gacggcctgc gcattgcacg caagctgctg ctggacatca acgattccgg tctgccagcc | 420 |
| gcaggcgagt tcctggatat gatcacccca cagtacctgg ccgatctgat gtcctggggt | 480 |
| gcaatcggtg ctcgcaccac tgaatcccag gtgcaccgcg aactggcatc cggtctgttc | 540 |
| tgcccagtgg gcttcaagaa cggcaccgac ggcaccatca aggtggcaat cgatgcaatc | 600 |
| aacgcagcag gcgcaccaca ctgctttctg tccgtgacca agtggggtca ctccgcaatc | 660 |
| gtgaacacct ccggcaacgg cgactgccac atcatccttc gcggtggcaa agaaccaaac | 720 |
| tactccgcaa agcacgtggc agaagtgaaa gaaggcctga acaaggctgg cctgccagca | 480 |
| caggtgatga tcgatttctc ccacgcaaac tcctccaagc agttcaaaaa gcagatggat | 840 |
| gtgtgcgcag atgtgtgcca gcagatcgca ggcggtgaaa aggcaatcat cggcgtgatg | 900 |
| gtggaatccc acctggtgga aggcaaccag tccttggaat ccggtgaacc actggcatac | 960 |
| ggcaagtcca tcaccgatgc ctgcatcggc tgggaagata ccgatgcact gctgcgccag | 1020 |
| ctcgcaaacg cagtgaaggc acgccgtggc taa | 1053 |

END

1. A recombinant cell of the genus Corynebacterium comprising a gene coding for a DAHP synthase as defined in SEQ ID NO. 11 or a variant thereof, with the proviso that, in the variant of the DAHP synthase, positions 76 and 211 remain unaltered.

2. The recombinant cell as claimed in claim 1, wherein in the variant of the DAHP synthase defined by SEQ ID NO. 11, positions 10, 13, 147, 148, 150, 151, 179, 209, 211 and 212 additionally remain unaltered.

3. The recombinant cell as claimed in claim 1, wherein said cell is C. glutamicum.

4. The recombinant cell as claimed in claim 1, additionally characterized by
(i) reduced activity of anthranilate phosphoribosyltransferase compared to the respective wild type, but where residual activity must be present; and
(ii) optionally the deletion or inactivation of the gene coding for phosphoenolpyruvate carboxylase as defined in SEQ ID NO. 4.

5. A process for producing at least one compound selected from the group consisting of oAB, 3-deoxyarabinoheptulosanate 7-phosphate, 3-dehydroquinate, 3-dehydroshikimate, shikimate, shikimate 3-phosphate, chorismate, prephenate, methylanthranilic acid, para-aminobenzoic acid, vanillin, indole, indoxyl, indigo, mandelic acid, ferulic acid and cinnamic acid comprising microbial fermentation of a recombinant cell comprising a gene coding for a DAHP synthase as defined in SEQ ID NO. 11 or a variant thereof, with the proviso that in the variant of DAHP synthase the positions 76 and 211 remain unaltered.

6. The process as claimed in claim 5, wherein the microbial cell is a Corynebacterium sp.

7. The process as claimed in claim 5, wherein the recombinant cell additionally differs from the wild type of the relevant microorganism by the following features:
(i) reduced activity of anthranilate phosphoribosyltransferase compared to the respective wild type, but where residual activity must be present; and
(ii) deletion or inactivation of the gene coding for phosphoenolpyruvate carboxylase as defined in SEQ ID NO. 4.

8. A process of producing ortho-aminobenzoic acid (oAB) comprising microbial fermentation of a 3-deoxyarabinoheptulosanate 7-phosphate synthase (DAHP synthase) having an amino acid sequence as defined by SEQ ID NO. 11 or a variant thereof, with the proviso that in the variant of the DAHP synthase positions 76 and 211 remain unaltered.

9. A process of producing oAB comprising:
a) providing an at least one recombinant microbial cell comprising a gene coding for a DAHP synthase as defined in SEQ ID NO. 11 or a variant thereof, with the proviso that in the variant of the DAHP synthase, positions 76 and 211 remain unaltered; and
b) culturing the at least one recombinant microbial cell in a culture medium comprising at least one fermentable carbon source, wherein oAB is produced.

10. The process as claimed in claim 9, wherein in process step b) a concentration of said compound of at least 1 g/L is achieved in the culture medium.

11. A composition comprising
a) at least one recombinant microbial cell comprising a gene coding for a DAHP synthase as defined in SEQ ID NO. 11 or a variant thereof, with the proviso that in the variant of the DAHP synthase, positions 76 and 211 remain unaltered;
b) at least one fermentable carbon source; and
c) at least one nitrogen source, at least one phosphorus source, at least one sulfur source and trace elements.

12. The composition as claimed in claim 11, additionally comprising at least one compound selected from the group consisting of oAB, 3-deoxyarabinoheptulosanate 7-phosphate, 3-dehydroquinate, 3-dehydroshikimate, shikimate, shikimate 3-phosphate, chorismate, prephenate, methylanthranilic acid, para-aminobenzoic acid, vanillin, indole, indoxyl, indigo, mandelic acid, ferulic acid and cinnamic acid.

13. The composition as claimed in claim 12, wherein the concentration of said compound is at least 1 g/L.
